(19) United States
(12) Patent Application Publication
(10) Pub. No.: US 2011/0171180 A1
(43) Pub. Date: Jul. 14, 2011

(54) BIOENGINEERED SKIN SUBSTITUTES

(75) Inventors: Katie A. Bush, Worcester, MA (US); George D. Pins, Holden, MA (US)

(73) Assignee: Worcester Polytechnic Institute, Worcester, MA (US)

(21) Appl. No.: 12/727,687

(22) Filed: Mar. 19, 2010

Related U.S. Application Data
(60) Provisional application No. 61/161,743, filed on Mar. 19, 2009.

(51) Int. Cl. A61K 35/12; C12N 5/071; A61P 17/02

(52) U.S. Cl. 424/93.7; 435/325

(57) ABSTRACT

A bioengineered skin substitute was developed that contains a microfabricated basal lamina analog that recapitulates the native microenvironment found at the dermal-epidermal junction (DEJ).

### RELATED APPLICATIONS

This application claims priority to co-pending U.S. Provisional Patent Application Ser. No. 61/161,743, filed Mar. 19, 2009, and entitled BIOENGINEERED SKIN SUBSTITUTES, which is incorporated by reference herein in its entirety for all purposes.

### STATEMENT REGARDING FEDERALLY SPONSORED RESEARCH OR DEVELOPMENT

This invention was made, in part, with Government support under grant numbers EB-005645 and P41 EB02503 awarded by the National Institutes of Health and grant number W81XWH-08-01-0422 awarded by the U.S. Army Medical Research and Material Command. The Government has certain rights in the invention.

### BACKGROUND OF THE INVENTION

In the development of bioengineered skin substitutes for replacement of skin lost to trauma or disease, the addition of biologically active molecules, that promote key events in non-scarring self-healing wounds, has the potential to guide epithelialization. In the native wound environment, fibronectin (FN) is part of the provisional matrix that interacts with dermal collagens and promotes the migration of keratinocytes through granulation tissue of the wound. Fibronectin is also involved in basement membrane synthesis and organization of the wound site, which are critical for the reestablishment of a healthy functional tissue. In vitro studies have examined the effect of FN on keratinocyte functions necessary for reepithelialization. When FN was passively adsorbed on bacteriological plastic, an increase in percentage of adherent cells was obtained. Studies where polystyrene was coated with FN showed enhanced migration and inhibition of terminal differentiation on the FN surfaces. Fibronectin also has been passively adsorbed to biomaterials that have the potential for implantation. Studies incorporating FN on the surface of PLGA, through passive adsorption, found limited keratinocyte migration; however, it was found that when FN was passively adsorbed to collagen, migration increased. Research investigating passive adsorption of FN to collagen-glycosaminoglycan (GAG) membranes found an increase in attachment over non-modified collagen surfaces.

In addition to investigating keratinocyte responses to full FN molecules, the modification of biomaterial surfaces with synthetic peptides located in the central cellular binding domain of FN, specifically the arginine-glycine-aspartic acid (RGD) sequence have been examined. Arginine-glycine-aspartic acid peptides have been covalently coupled to collagen-GAG matrices and to a hyaluronate synthetic matrix. Both studies found increased keratinocyte attachment and spreading in comparison to those on unmodified matrices. Although this approach allows for more RGD sites to be expressed on the surface of the biomaterials, these short sequences lack full biological activity when compared with the native protein.

During wound healing, as well as in cell culture expansion from healthy skin, keratinocytes express an increase in the integrin receptor α5β1 which is specific for the central cellular binding domain of FN. The availability of this FN domain and its full biological activity is highly dependent on the structural orientation of the protein and has been found to be critical in modulating cellular functions. When FN adsorbs to a surface, it undergoes a conformational change, which is highly dependent upon the properties of the surface. Recently, the availability of the central cellular binding domain of FN and its role on keratinocyte morphology, attachment, and differentiation was investigated using self-assembled monolayers as model biomaterial surfaces. A direct relationship was found between keratinocyte spreading area and attachment, and an indirect relationship was found between cellular binding domain availability and cell differentiation. When evaluating focal adhesion formation, it was found that the area density of focal adhesions in individual keratinocytes directly corresponded with the availability of the central cellular binding domain of FN, suggesting that the functions evaluated were integrin mediated.

Bioengineered skin substitutes offer a promising approach in the treatment of severe burns or chronic wounds when autografts are not an option for the patient. Clinically, these substitutes provide a barrier to prevent infection and water loss as well as therapeutic effects that induce dermal tissue repair and stimulate healing of chronic wounds. Although there has been clinical success with these grafts, limitations still exist including prolonged healing times, mechanically induced graft failure, poor graft take, and residual scarring. Additionally, current bioengineered skin substitutes only restore a subset of anatomical structures that play key roles in normal physiological functions of skin.

One design feature common to current bioengineered skin substitutes is a flat interface between the dermal and epidermal components. At the dermal-epidermal junction (DEJ) of native skin there is a basal lamina which contributes critical cues involved in regulating keratinocyte functions necessary for the maintenance of the tissue architecture, as well as skin's overall homeostasis with the surrounding environment. The basal lamina is a thin membranous sheet composed of both collagenous and non-collagenous extracellular matrix (ECM) proteins including type TV collagen (CIV), laminin (LN), fibronectin (FN), and heparin sulfate proteoglycans. The basal lamina is not flat, but rather conforms to a series of three dimensional ridges and invaginations formed by papillae located in the upper region of the dermis that range from 50-400 μm in width and 50-200 μm in depth. It has been determined that in different regions of the body, the number and dimensions of dermal papillae and rete ridges differ. In areas of skin exposed to excessive friction, such as the palms and soles, the basal lamina conforms to a series of longer and more numerous dermal papillae and deeper rete ridges, suggesting that the increased surface area provided by the topographical features also aids in enhancing mechanical stability.

Keratinocytes in direct contact with the basal lamina are the only population of cells in the epidermis with the capacity to proliferate. The epidermis is in constant renewal, thus proliferation is necessary in order to provide proper barrier function. The population of proliferating basal keratinocytes is heterogeneous and contains epidermal stem cells (ESCs) and transit amplifying (TA) cells that have different regenerative and differentiation capabilities. Epidermal stem cells are non-differentiated cells that are responsible for the assembly and maintenance of the epidermis as well as the rapid regeneration of damaged tissue. They are capable of self-renewal and give rise to TA cells, which divide a finite number of times to amplify the basal layer and then undergo terminal differentiation.

Epidermal stem cells exhibit a high degree of spatial organization and clustering along the complex topography of the basal lamina. Epidermal stem cells can be further classified based on their localization into bulge ESCs, found in the bulge region of the hair follicle and interfollicular ESCs found either in the bottom of rete ridges or tips of papillary projections. Several studies have examined the localization of proliferating keratinocytes and interfollicular ESCs in the basal layer of native epidermis using cell cycling or integrin detection techniques. In monkey palm epidermis, DNA label-retaining cells (LRCs) were found in the deep rete ridges; which is indicative of slowly cycling cells, a well accepted characteristic of ESCs. This cell-cycle kinetic analysis has been used to investigate the localization of ESC populations in other species and tissue sites such as hamster epidermis and oral mucosa, the bulge region in hair follicles, and human embryonic and fetal epidermis. In addition to label retaining cells, research has been conducted evaluating the intensity of β₁ integrin receptors and correlating the findings with interfollicular ESC localization. All basal keratinocytes express β₁ which mediates adhesion to the ECM of the basal lamina and regulates terminal differentiation. Enhanced β₁ expression has been found to distinguish ESCs from keratinocytes with lower proliferative potential. The expression of β₁ has been found to be distributed differently along the microtopography of the basal lamina, based on body site location. These findings correspond with label-retaining experiments previously mentioned. In human skin, β1-bright regions are found in deep rete ridges in the palms and soles; whereas in interfollicular epidermis of the scalp, foreskin, and breast, β₁-bright regions were found at the tips of the papillary projections.

In addition to studies evaluating microtopographic features of the basal lamina in native tissues and interfollicular ESC localization, other researchers have focused their efforts on investigating the effects of the biochemical composition of the basal lamina that influences keratinocyte attachment and ESC selection, proliferation, and terminal differentiation. Keratinocyte attachment was investigated on CI, CIV, LN, and FN at varying concentrations and amounts of time. It was determined that the percentage of keratinocytes that adhered to each surface was time dependent as well as ECM protein and concentration dependent with adhesion to FN giving the highest percentage of adherent cells. Studies have also investigated the ability to select for ESCs based on using rapid adhesion assays on ECM proteins. Differences in colony forming efficiency (CFE), a metric that can be used to demonstrate the presence of an ESC population or proliferative potential of the population, have been detected based on this selection technique. Additionally, flow cytometry has been used to sort keratinocytes based on β₁ integrin expression. When evaluating the CFE of keratinocytes separated using this technique, a linear relationship was found between log fluorescence and CFE, which implies a log linear relationship between β₁ integrin density on the cell surface and proliferative potential. Studies have further examined the effects of ECM proteins of the basal lamina, specifically FN, on differentiation of keratinocytes. It has been shown that when cells are induced to undergo differentiation in culture, they become less adhesive to FN, and no longer express the β₁ integrin.

### BRIEF SUMMARY OF THE INVENTION

The invention is directed to a skin substitute of a basal lamina analog comprising extracellular matrix protein, a dermal sponge and keratinocytes. The extracellular matrix protein is selected from the group consisting of collagen I, collagen IV, fibronectin, laminin, glycosaminoglycan and combinations thereof. Fibronectin can be covalently bound to collagen I, collagen IV or collagen-glycosaminoglycan. The fibronectin can be covalently bound using a chemical crosslinking agent such as 1-ethyl-3-(3-dimethylaminopropyl)carbodiimide hydrochloride.

The invention if also directed to a method of making a skin substitute by creating a master pattern containing channels on a silicon wafer; casting polydimethylsiloxane onto the silicon wafer; allowing the polydimethylsiloxane to polymerize; casting a first extracellular matrix protein onto the polymerized polydimethylsiloxane; allowing the first extracellular matrix protein to polymerize; casting a second extracellular matrix protein onto the back surface of the first extracellular matrix protein; attaching an extracellular matrix protein sponge to the back surface of the first extracellular matrix protein; chemically crosslinking the first extracellular matrix protein, second extracellular matrix protein and extracellular matrix protein sponge to form a composite; removing the composite from the polymerized polydimethylsiloxane; conjugating fibronectin to the front surface of the composite; sterilizing the composite; and adding keratinocytes. The first and second extracellular matrix protein can be collagen, and particularly collagen type I. The extracellular matrix protein sponge can be collagen-glycosaminoglycan.

The invention also provides a method of treating wounds or burns by administering a skin substitute comprising a basal lamina analog comprising extracellular matrix protein, a dermal sponge and keratinocytes.

To improve the regenerative capacity of biomaterials scaffolds, biomolecules have been incorporated to present biochemical cues that direct cellular functions. This approach requires that the biomolecules are precisely tailored to the surface of the biomaterial to ensure that the appropriate cellular binding domains are presented for maximum bioactivity. To improve the compatibility and regenerative potential of biomaterials scaffolds, FN is a protein of interest to adsorb to the surfaces, based on its role in cell adhesions, migration, and differentiation. However, several studies indicate that when FN is passively adsorbed to the surface of biomaterials, its conformation is effected by surface properties, which modulate cellular binding site presentation as well as biological activity. This invention relates, in part, to the effect of passive adsorption of FN on epithelialization of a basal lamina analog. Additionally the presentation sites of the central cellular binding domain of FN were evaluated based on the preparation technique of the basal lamina analog and the conjugation strategy. Overall it was determined that EDC conjugation of FN to the surface of self-assembled CI membranes improved binding site availability.

Fibronectin enhanced epithelial thickness and keratinocyte proliferation on the surfaces of collagen-GAG basal lamina analogs. When FN was passively adsorbed at a saturation density previously determined on the surface, epithelial thickness was enhanced in comparison to untreated membranes at both 3 and 7 days of A/L interface culture. The morphology of basal keratinocytes on the FN grafts exhibited a more native columnar morphology than those on the scaffolds without FN. When keratinocyte proliferation was examined using Ki67, a nuclear marker for proliferation, it was found that the percentage of Ki67 positive cells at 3 days of A/L interface culture on FN treated membranes was greater than on untreated membranes, ˜35% to ˜20% of total basal cells, respectively. At 7 days of A/L interface culture, no differences were found between percentages of Ki67 positive basal keratinocytes, with both membranes having ˜20% of total basal cells.

In unwounded epidermis, between 10 and 20% of basal keratinocytes are proliferative, based on the location of the skin. In an acute wound environment, keratinocyte proliferation is increased. Within hours after injury, keratinocytes at the wound edge become activated and undergo a phenotypic change which facilitates migration over the wound bed. A proliferative burst occurs 24 to 72 hours post injury and after wound closure, the proliferative capacity of the basal layer returns to normal status. This invention includes FN treated scaffolds that closely mimic the wound environment, and provide the appropriate signals for proliferation to occur. Once the cells sense that a monolayer is formed, proliferation returns to normal and the cells begin to undergo differentiation and migrate upward to create a stratum corneum that provides protection from the environment.

The presentation of the cellular binding domain of FN that was passively adsorbed on the collagen-GAG basal lamina analogs was investigated after evaluating the effects of FN on graft morphology and proliferation. It was determined that the FN cellular binding site presentation directly corresponded with previously published values for keratinocyte attachment to collagen-GAG membranes. It was concluded that the collagen-GAG membrane surfaces were being saturated using passive adsorption since there were no differences between membranes that were treated with 100 μg/ml or 300 μg/ml of FN. To increase the number of FN presentation sites, different sources of collagen were evaluated to fabricate membranes as well as conjugation strategies to covalently link FN to the surfaces.

In this invention, the presentation of FN cellular binding domains on collagen-GAG basal lamina analogs was analyzed and compared with the FN cellular binding domains on self-assembled CI basal lamina analogs. Initially, collagen-GAG membranes fabricated from an FDA approved, commercially available product were used to facilitate a rapid translation from benchtop to bedside. Although this product has many advantageous properties; the starting collagen material is considered “insoluble” when placed in an acidic environment and does not completely dissolve into individual collagen fibrils. When a suspension of these collagen fibrils is air-dried, the aggregates of fibrils come together and form a membrane with random orientation. In contrast, the self-assembled CI membranes developed for this invention are fabricated from a solution of acid solution type I collagen molecules. When neutralized, these collagen molecules self-assemble into individual fibrils, and aggregate laterally and linearly to form collagen fibers with structural morphology comparable to native tissue constructs. This invention shows that when 100 μg/ml of FN is passively adsorbed to the surfaces of the different collagen membranes, the self-assembled CI basal lamina analog has significantly more FN cellular binding site availability than the collagen-GAG basal lamina analog. With CI, the FN binding site is found on the α1(1) chain between amino acid residues 757-791. When the soluble collagen self-assembles it exposes the FN binding site, similar to that in native tissue, unlike the collagen-GAG fibers that do not have all FN binding sites exposed, because of the random packing of the fibrillar aggregates. Additional analysis was performed evaluating the cellular binding site availability of FN on self-assembled CI basal lamina analogs at varying concentrations of FN to determine the saturation limit. It was found that the availability of FN on the surfaces of the self-assembled CI membranes at 100 ng/ml of FN was the optimal concentration for binding site availability, similar to the evaluation of binding site availability on collagen-GAG membranes.

Various investigations have evaluated covalent conjugation strategies to improve the presentation and bioactivity of FN over passive adsorption on various surfaces. The use of a carbodiimide conjugation strategy was evaluated to crosslink the membranes as well as to covalently bind FN. This crosslinking agent has been highly successful in crosslinking collagen and improving its degradation resistance and mechanical properties, as well as coupling chondroitin sulfate, heparin sulfate, and heparin to the surface of collagen scaffolds. The current invention relates, in part, to a method to covalently conjugate FN to the surface of both collagen based scaffolds resulting in a significant increase in cellular binding site availability of FN when compared to that of using passive adsorption. When keratinocytes were cultured at 3 days at the A/L interface on self-assembled basal lamina analogs with no FN, passively adsorbed FN, and EDC conjugated FN, an increase in epithelial thickness was found between all surfaces. This data also corresponds with the data from the FN cellular binding domain availability analysis. Overall the results from these studies indicate that the cellular binding domain of FN can be enhanced on collagen-based biomaterials and directly influences functions important for epithelialization. The information gained from this invention can be applied to other model systems where the enhancement of cellular binding sites of FN on collagenous biomaterials would enhance tissue functionality. Additionally this information can be used in the design of engineered tissues where the incorporation of a basal lamina analog is necessary to direct epithelial polarity and functions as well as to separate cell types and act as a selectively permeable barrier, such as in the glomerulus of the kidney or the small intestine.

Understanding how the biochemical and three-dimensional microenvironment of the basal lamina modulates keratinocyte proliferation and differentiation, as well as contributes to localization of ESCs, is of great importance when designing bioengineered skin substitutes. In native tissues, the basal lamina provides instructive cues that are critical in skin architecture, cellular organization, and the regeneration of the epidermal layer. The regeneration of skin is of great importance, because in order for skin to provide the protective barrier against the surrounding environment, the epidermis must be in constant renewal. In this invention a novel dermal scaffold was developed that contains both biochemical and microtopographical cues provided by the native basal lamina and the role of the microenvironment on bioengineered skin substitutes morphology, epidermal thickness, keratinocyte proliferation, and ESC localization was investigated. Additionally the findings were compared with epithelialized DED and native foreskin tissues.

To create a microfabricated basal lamina analog produced from self-assembled CI, photolithography was used. A master pattern was created on a silicon wafer to produce channels with specified features of 200 μm depth and 50 μm, 100 μm, 200 μm, and 400 μm widths. A negative replicate was produced using PDMS and acid soluble type I collagen was self-assembled on the surface of the negative replicate PDMS pattern. Previously, a similar strategy was used to create basal lamina analogs using a collagen-GAG coprecipitate with different processing techniques to create a basal lamina analog laminated to a dermal scaffold. When comparing the two strategies to produce microfabricated basal lamina analogs, it was found that the features of the microfabricated basal laminas when composed of collagen-GAG had a greater error associated with both the widths and depths (mean width error varied from 13-30% and mean depth varied from 7.4-16.2%), than the features on the self-assembled CI lamina analogs (mean width errors varied from 2-9% and mean depths varied from 0.9-2.5%). Although the depths and widths of the self-assembled CI membranes varied from the design specifications, the method of the invention using self-assembled CI demonstrates improved fidelity for recapitulating topographical features as well as a defined starting biochemistry for enhanced FN EDC conjugation.

After analyzing the topography of the channels, the responses of keratinocytes cultured for 3 or 7 days at the A/L interface on the surfaces of microfabricated basal lamina analogs laminated to dermal scaffold were investigated and the results were compared to keratinocytes cultured on DED as well as with native neonatal foreskin and adult breast tissue. When evaluating histological images, it was determined that the epidermal thickness varied based on the geometry of the channels. It was also determined that after culturing keratinocytes on the microfabricated basal lamina analogs, that the topographic features had greater errors associated with their dimensions, than when measured prior to cellular culture. Therefore to account for the change in channel width, only channels with widths that deviated from the mean by +/−2 SEM were analyzed, and normalized the epidermal thickness values to the depths of the channel based on previous data that suggests depth plays a role in the microenvironment.

The observed changes in topographical features of the epithelialized microfabricated self-assembled CI basal lamina analog can be explained based on in vivo analysis of MMPs in normal wound healing. Matrix metalloproteinases (MMPs) are found in the wound environment and are responsible for the degradation and modification of ECM proteins at the wound site. Matrix metalloproteinase-1 (MMP-1), or collagenase-1, is keratinocyte derived and initially found at high levels in the wound to enable keratinocyte migration and monolayer formation. Once a monolayer and basement membrane proteins are formed, this enzyme ceases (as well as other MMPs) to be produced at high levels, and returns to normal levels that contribute to the constant balance of matrix synthesis and breakdown and recycling of the ECM. Since the keratinocytes initially seeded on the microfabricated basal lamina analogs exhibit similar characteristics to wounded keratinocytes, it is hypothesized that there was an upregulation of MMP levels similar to in vivo wounds which caused the change in the dimensions of the topographic features.

The epidermal layer of bioengineered skin substitutes was evaluated after 3 days of A/L interface culture, and it was determined that keratinocytes cultured in 50 μm width channels had statistically similar epidermal thickness values as epithelialized DED. At 7 days of A/L interface culture the 50 μm and 100 μm width channels exhibited the same epidermal thicknesses as keratinocyte cultured on DED and foreskin tissue and these conditions were statistically different from epidermal thickness values in the 200 μm width and 400 μm width channels.

The morphology of the epidermal layer on the FN conjugated basal lamina analog surfaces, suggests well differentiated epidermal layers, based on cellular size and loss of nuclei from the stratum corneum layer. Keratinocytes found in the basal layer are cuboidal in shape and as the cells progress to the stratum corneum, exhibit a more flattened morphology, similar to what is found in native skin. Furthermore, in native skin, these morphological changes are accompanied by changes in the expression of keratin proteins and water proofing lipids, which are both important in functionality of the skin in providing a protective barrier against the environment as well as structural integrity of the epidermis.

Additionally, the percentage of Ki67 positive basal keratinocytes was determined to demonstrate functionality of our bioengineered skin substitute. Native skin is under constant renewal, thus having a bioengineered skin substitute with similar regenerative capacity is necessary in order to maintain a healthy tissue. Ki67 positive basal keratinocytes were measured at the 3 and 7 day time points. At 3 days of A/L interface culture, the 50 μm width channels contained a lower percentage of Ki67 positive basal cells than any other channels and was similar to the percentage of Ki67 basal keratinocytes on DED. At 7 days of A/L interface culture, the 200 μm width and 400 μm width channels had displayed a decrease in percentage of Ki67 positive basal keratinocytes, whereas the 50 μm width and 100 μm width channels stayed relatively consistent.

The data obtained from our Ki67 analysis helps to elucidate the trends from the epithelial thickness experiments and indicates that a space filling mechanism can be used to explain the data. The data indicates that after initial seeding, a monolayer of cells was present and that a proliferative burst occurred, similar to results seen during in vitro cultures of low-density to high-density keratinocytes as well as in the in vivo wound healing environment once a monolayer of keratinocytes is formed and contact inhibition occurs. This burst can be characterized by the basal cells undergoing two to four mitotic divisions and committing to terminal differentiation that leads to epithelialization. Since the 50 μm width channels have much smaller dimensions, they require a fewer number of cells to fill the topographic feature, followed by the 100, 200, and 400 μm width channels. At 3 days of A/L interface culture (6 days of culture); the 50 μm width channels had a complete epithelial layer; however the 100, 200, and 400 μm width channels did not. The Ki67 data suggests that a proliferative burst occurred before the 3 days time point and this channel was in a steady state of proliferation between 3 and 7 day time points, whereas the other channels were still undergoing a proliferative burst to fill the channel. At 7 days of A/L interface culture (10 days of culture); the 100 μm width channels had the same epithelial thickness as the 50 μm width channels and native skin; however the 200 μm and 400 μm width channels contained a less thick epidermis. The percentage of Ki67 positive cells for the 200 μm and 400 μm width channels both decreased at the 7 day time point but were not statistically different from the 3 days, which could indicate that the epithelial thickness in these channels was as thick as it would form.

Although the presence of Ki67, a marker for proliferative cells, was evaluated, this marker does not distinguish between the two types of proliferating cells, ESCs and TAs, found in the basal layer of the epidermis. In order to create a bioengineered skin substitute that has the capacity for continuous renewal, it is necessary for ESCs to be present on the surface of the bioengineered skin substitute. In the basal layer of the epidermis, keratinocytes express receptors of the integrin family that mediate adhesion to the basal lamina and also regulate the onset of terminal differentiation. Adhesion to ECM proteins and fluorescence activated cell sorting (FACS) have both been used to separate basal keratinocytes based on their integrin expression levels. When plating the separated fractions of keratinocytes and examining CFE, the cells expressing a two- to threefold increase in β₁ levels were determined to have greater proliferative potential. Additionally when using fluorescence microscopy, the location of β₁-bright regions in native tissues was compared with the location of LRCs from previous studies, and it was found that they both resided in the same location, which was based upon tissue site. The presence of β₁ in colonies of cultured keratinocytes was investigated and it was determined that 25% of cells in the colony were β₁-bright and these cells were located at the colony border. This data corresponds with previously published literature that selected for keratinocytes using rapid adhesion to CIV. In this invention the keratinocytes that adhered were 28% of the total starting population and had a higher modal α₂β1 fluorescence than the total (unselected) basal population. This keratinocyte population is important because this is the starting population of cells to be cultured on the surface of a dermal scaffold with a microfabricated basal lamina analog.

Immunofluorescent microscopy and image analysis of sections of the grafts was utilized to evaluate the location of these β₁-bright cells on bioengineered skin substitutes. For our bioengineered skin substitutes, we found that the β₁-bright regions were located in the channels and not on the papillary plateaus. Analyses indicated that for the 100 μm width channel, 16.7% of the total basal keratinocyte population in the channel was β₁-bright. Similar analysis for the 400 μm width channel indicated that 23% of the total basal keratinocyte population in the channel was β₁-bright. Additionally it was found that the β₁-bright regions in the 400 μm width channels localized to the corners of the channels as seen in FIGS. 19G and 19H When just evaluating the “corner” regions of the 400 μm width channels it was found that 50% of the basal keratinocytes in this region were β₁-bright. When evaluating the papillary plateaus, it was found that there were no β₁ bright cells (0%). When flat regions of the bioengineered skin substitutes were evaluated, it was found that the β₁-bright cells were heterogeneously dispersed and that 30% of the total basal keratinocyte population was β₁ bright. For epithelialized DED we found that 15.6% of the total basal keratinocyte population was β₁-bright and these cells were localized to the rete ridges. In native foreskin tissue, it was found that 7% of the total population of basal keratinocytes was β₁-bright and these cells were localized to the tips of the dermal papillae. This localization finding is consistent with literature; although the percentage of integrin bright cells was much lower. This could be caused by the variation of fluorescence intensities that the samples were exposed to. In this invention, care was taken to not overexpose the regions, thus lower values could be caused by this factor.

In addition to identifying ESCs in bioengineered skin substitutes, an interesting finding is that the β₁-bright cells were found primarily in the channels as well as in the rete ridges of epithelialized DED. Also the current analysis of β₁ in foreskin tissue is consistent with previous studies indicating that β₁-bright regions are localized to the tips of the papillary projections. In native skin the localization of integrin bright regions varies with location in the body. This localization may be a mechanism to protect the cells that contribute to the maintenance of population of cells responsible for the continuous regeneration of the skin. There are many insults that can occur from the outside environment such as ultraviolet light or chemicals, which would make the deep rete ridges a more protective microenvironment for the ESCs, however insults can also occur from the dermal tissue as well. Inflammation or a burst of oxidative stress could damage the cells in the bottom of the rete ridges and therefore the safer place would be in the tips of the papillary projects. Neither of these groups of insults explains why in one location of the body, the ESCs in skin would be in the bottom of the rete ridges or in the tips since all insults mentioned can occur in all locations of the body. Another possible explanation for the localization of ESCs is based on the occurrence of mechanical friction in different regions of the body. The palms and soles of the human body are areas of skin that are exposed to excessive friction and contain more numerous dermal papillae and deep rete ridges. When investigating β₁ expression in these tissues, it was found that the bright regions are in the deep rete ridges, unlike other areas of the body that experience less friction and have β₁-bright expression on the tips of the papillary projections.

A similar range of percentages of β₁-bright basal keratinocytes was found to correspond with previous literature in suggesting that 25-50% of basal keratinocyte are β₁-bright. However, other analyses suggest that only 10% of basal keratinocytes are ESCs and another report suggest a much lower percentage (1%) of the basal cells are actually ESCs. Quantitative differences in the expression of one particular cell surface marker is not sufficient to uniquely define the stem cell population, since β₁ is not unique to ESCs. Consequently, the analysis of the effect of the microenvironment on ESC localization, necessitates that future studies investigate additional means of interfollicular ESC detection. However, there is no universally accepted criterion to define interfollicular ESCs, and surface markers used to isolate a population may not isolate a distinct population, but one that has overlapping population of cells. Until a detection technique is discovered, it will be necessary to compile evidence of “sternness” combining many techniques such as the evaluation of the expression of β₁, transferrin receptors, connexin 43, isoform of CD133, desmosomal proteins, and proteins mediating intercellular adhesions, as well as label retaining studies. Additionally, studies evaluating the transcriptional profiles of cells isolated using surface markers will have an impact on identifying a true interfollicular ESC population.

Overall this invention has focused on developing a bioengineered skin substitute that recapitulates biochemical and microtopographical features found at the DEJ to enhance epithelialization and interfollicular ESC localization. It was found that 50 and 100 μm width channels with approximate depths of 150 μm contain a full epithelial layer after 7 days at A/L interface culture. When comparing these values to epithelialized DED or native skin, it was found that the epithelial thicknesses were not statistically different from one another and also contain similar values of proliferating basal keratinocytes. Additionally, the bioengineered skin of the invention substitute containing a microtopographical basal lamina analog provides an excellent model system to evaluate the proper ESC niche through both surface markers and label-retaining studies in order to enhance the regenerative capacity of bioengineered skin substitutes.

### BRIEF DESCRIPTION OF THE DRAWINGS

FIG. 1A is a Computer-Aided Design (CAD) drawing of individual parts of a Custom Built Air Liquid Interface (A/L) Culture Device including the base and top pieces with posts on the base piece to allow for alignment of the two pieces and initial stability. A screen sits on the base piece that the membrane is placed on. This screen facilitates diffusion of cell culture medium from below the membrane and A/L interface culture. A silicone o-ring is fit in the base piece to provide a tight seal that creates a well on the surface of the collagen membrane that allows for protein modification and cell seeding. The complete unit fits in a 6-well plate.

FIG. 1B is a CAD drawing of the A/L Interface Culture Device with base and top piece screwed together.

FIG. 1C is a photograph of the A/L Interface Culture Device with a collagen membrane placed on top of the screen during assembly.

FIG. 2 is photographs of Histological Representations of the Thicknesses of Epidermal Layers on Collagen-GAG Membranes. Keratinocytes were cultured for 3 or 7 days at the A/L interface on collagen-GAG control (non-modified) membranes or collagen-GAG membranes that were modified by passively adsorbing FN to the surfaces of the scaffolds. At 3 or 7 days of A/L interface culture the thickness of the epithelial layer on collagen-GAG membranes treated with FN was greater than that on untreated collagen-GAG membranes. Scale bar represents 30

FIG. 3 is a bar graph of the Quantitative Evaluation of Epidermal Thickness on Collagen-GAG Membranes. The thicknesses of the epithelial layers at 3 or 7 days of A/L interface culture were measured on control (non-modified) collagen-GAG membranes or collagen-GAG membranes that were modified by passively adsorbing FN to the surfaces. At both 3 and 7 days there was a significant difference between untreated and FN treated surfaces. (* indicates p<0.05 Student's t-test) Samples for 3 day culture are n=7 and for 7 day culture n=4.

FIG. 4 is photographs of Histological Representations of Ki67 Positive Keratinocytes on Collagen-GAG Membranes. Keratinocytes were cultured on collagen-GAG membranes for 3 or 7 days at the A/L interface on control (non-modified) collagen-GAG membranes or collagen-GAG membranes modified by passively adsorbing FN to the surfaces. At 3 or 7 days of A/L interface culture Ki67 immunostaining (brown stained nuclei) was used to evaluate proliferation of basal keratinocytes. Scale bar represents 30 μm.

FIG. 5 is a bar graph of the Quantitative Analyses of Ki67 Positive Basal Keratinocytes on Collagen-GAG Membranes. The percentage of positive Ki67 basal keratinocytes at 3 or 7 days of A/L interface culture was measured on control (non-modified) collagen-GAG membranes or collagen-GAG membranes modified by passively adsorbing FN to the surfaces. At 3 days statistical differences were found between keratinocytes cultured on control surface and FN treated surfaces (* indicates p<0.05 Student's t-test). For all experimental conditions, n=5 samples were measured at both 3 and 7 days of culture.

FIG. 6 is a bar graph of the Availability of Cellular Binding Domains for FN Passively Adsorbed on Collagen-GAG Basal Lamina Analogs. The availability of the cellular binding domain of FN on collagen-GAG basal lamina analogs was evaluated using a quantitative immunofluorescent assay. Fibronectin concentrations of 0, 30, 100, and 300 μg/l were evaluated and it was determined that at a concentration of 100 μg/ml the average RFI was statistically different from 0, and 300 μg/ml, but did not statistically differ from 300 μg/ml indicating that a saturation plateau was achieved. Data is reported as averages and error bars indicate standard error mean with n=3. (*Indicates statistical difference, one way Analysis of Variance (ANOVA) with Tukey post hoc analysis p<0.05.)

FIG. 7 is a Schematic Representation of EDC-Mediated Conjugation of FN to Collagen. The carbodiimide, 1-ethyl-3-(3-dimethylaminopropyl)carbodiimide hydrochloride (EDC), was added to basal lamina analogs at a 5:1 molar ratio (EDC molecules to carboxylic acids in collagen). The EDC reacts with a carboxylic acid from the collagen molecule to form an unstable reactive o-acylisourea ester that can either couple with an amine group in collagen from the basal lamina analog or an amide group in FN that is added to the collagen membrane. If the unstable reactive o-acylisourea ester does not interact with an amine, it hydrolyzes and the carboxyl group is regenerated, thus returning back to its native state.

FIG. 8 is a bar graph of the Availability of Cellular Binding Domain of FN on assembled Type I Collagen (CI) membranes, a collagen-GAG and Self-Assembled CI Basal Lamina Analogs. A quantitative immunofluorescent assay was used to measure the availability of the cellular binding domain of FN on the surfaces of modified collagen membranes. * indicates p<0.05, Student's t-test and ** indicates p<0.05, One Way ANOVA with Tukey post hoc analysis. For collagen-GAG membranes, n =8 for passive adsorption and EDC conjugation. For self-assembled CI membranes n=4 for 30 and 300 μg/ml for both passive and EDC and n=8 for 100 μg/ml for both passive and EDC. Experiments were repeated and similar trends were we compared the binding found.

FIG. 9 is photographs of Low and High Magnification Histological Images of Self-Assembled CI Basal Lamina Analogs Treated with FN. The surfaces of self-assembled CI basal lamina analogs were treated with dPBS (controls) (A and D), passive adsorption of FN (B and E), or EDC conjugation of FN (C and F) and keratinocytes were cultured on the membranes at the A/L interface for 3 days. Conjugation of FN to the surfaces of the self-assembled CI basal lamina analogs using EDC caused an increase in epidermal thickness in comparison to control interface to surfaces and surfaces treated by passively adsorbing FN to the scaffolds. Scale determine whether bars represent 50 μm.

FIG. 10 is a bar graph of the EDC Conjugation of FN on Self-Assembled CI Basal Lamina Analogs Enhances Epidermal Thickness. Self-assembled CI basal lamina analogs were prepared and the surfaces were treated with dPBS (Control), passive adsorption of FN (Passive Adsorption), EDC conjugation of FN (EDC Conjugation) and keratinocytes were cultured at the A/L interface for 3 days. All surfaces were statistically different from each other. Surfaces treated with EDC conjugation of FN exhibited the greatest epidermal thickness values (* indicates p<0.05, One-Way ANOVA with Tukey post-hoc analysis). Bars indicate mean values and error bars are standard error and sample numbers are n=3 for control and passive adsorption and n=5 for EDC conjugation.

FIG. 11 is a schematic diagram of the Production of Bioengineered Skin Substitute Containing Microfabricated Basal Lamina Analogs. Photolithography (A) was used to create a master pattern on a silicon wafer containing channels with a depth of 200 μm and widths of 50, 100, 200, and 400 μm. Polydimethylsiloxane (PDMS) was cast on the microfabricated silicon wafer (B) and allowed to polymerize. The PDMS pattern was inverted and a collagen gel was cast onto the surface containing the negative replicate of the original pattern (C). Once polymerized, another collagen gel was cast onto the back surface of the original collagen gel which acts as a glue to laminate a collagen-GAG sponge (D). This composite was EDC crosslinked (E). The composite was removed from the PDMS and FN was conjugated to the surface (F). The composite was sterilized, seeded with keratinocytes (G), and cultured to create an engineered graft with a stratified epidermis.

FIG. 12A is a photograph of a histological section stained with eosin. Topographical Measurements of the Surfaces of Bioengineered Basal Lamina Analogs. To determine the dimensions of basal lamina analogs created using photolithography, histological sections were analyzed. A) represents a section stained with eosin. The insert illustrates the measurements made for depths (D) and widths (W) of the channels as well as the papillary plateau (PP) which will be discussed in later sections.

FIGS. 12B and 12C are bar graphs of the topographical measurements of the surfaces of bioengineered basal lamina analogs. All channels in the bioengineered skin substitutes were measured. These values were averaged and plotted in B (width) and in C (depth) against specified channel widths. Values are reported as averages +/±SEM. Sample numbers for the 50 μm width channels are n=4 and for the 100, 200, and 400 μm width channels n=5.

FIG. 13 is photographs of Histological Representation of Hematoxylin and Eosin Stained Bioengineered Skin Substitutes. To evaluate the effects of FN and topography on epithelialization, the epidermal thickness of the composite was measured without FN cultured for 3 days at the A/L interface (A and B), on composites with FN cultured for 3 or 7 days at the A/L interface (C and D, and E and F, respectively) and compared to keratinocytes cultured on DED cultured for 3 or 7 days at the A/L interface (G and H, respectively) and foreskin and breast control tissues (I and J, respectively). Composites without FN lacked a continuous layer of keratinocytes in all regions and only contained 1 to 3 cellular layers as well as cellular debris. Cells cultured on scaffolds containing FN had a continuous monolayer and comparable epidermal thicknesses and morphology to epithelial layers on DED and in native tissues. Scale bars=50 μm.

FIG. 14 is bar graphs of the Epidermal Thickness of Bioengineered Skin Substitutes Normalized to Depth of Channel. Epidermal thickness was measured in each channel of each composite and normalized to the depth of the channel. A) At 3 days of A/L interface culture, epidermal thicknesses measured in 50 μm channels were statistically increased over that of all other channels measured (* indicates p<0.05, One-Way ANOVA, Tukey post-hoc analysis). B) When evaluating epidermal thicknesses at 7 days of A/L interface culture the 50 μm width channels and the 100 μm width channels were statistically different than the 200 and 400 μm channels (* indicates p<0.05, One-Way ANOVA, Tukey post-hoc analysis. Large dashed lines represent epidermal thickness of foreskin tissue and smaller dashed lines represent epidermal thickness on DED. Values represent means +/−SEM. Samples for 50 μm and 100 μm widths at 3 and 7 days are n=5 and for the 200 μm widths n=6 and 15 at 3 and 7 days, respective n=11 and 13 for 400 μm channels at 3 and 7 days, respectively.

FIG. 15 is a bar graph of the Epidermal Thickness at Papillary Plateau. The thicknesses of the epidermal layers at the papillary plateau of bioengineered skin substitutes, epithelialized DED, and in native foreskin were measured. The dashed line represents the epithelial thickness of foreskin tissue. No statistical differences were detected between the thicknesses of bioengineered skin substitutes and epithelialized decellularized dermis at 3 or 7 days of A/L interface culture (One-Way ANOVA with Tukey post-hoc analysis). At 7 days there were no statistical differences between foreskin tissue (dashed line) and either the bioengineered skin substitutes or epithelialized decellularized dermis (Kruskal-Wallis One-Way ANOVA on Ranks). Values represent mean +/−SEM. For bioengineered skin grafts n=14 and 15 at 3 and 7 days, respectively, n=4 and 7 for epithelialized DED at 3 and 7 days, respectively, and n=4 for foreskin tissues.

FIG. 16 is photographs of Histological Representation of Ki67 Expression of Basal Keratinocytes Present in Bioengineered Skin Substitutes. To evaluate the effects of topography on the presence of proliferating basal keratinocytes, Ki67, a marker for highly mitotic cells was used. The presence of Ki67 positive basal keratinocytes was evaluated on bioengineered skin substitutes. A and B represent channels with 50 μm widths at 3 and 7 days, respectively. C and D represent channels with 100 μm widths at 3 and 7 days, respectively. E and F represent channels with 200 μm widths at 3 and 7 days, respectively. G and H represent channels with 400 μm widths at 3 and 7 days respectively. I and J represent epithelialized DED at 3 and 7 days. K and L are foreskin and breast tissue. Scale bars in all images=100 μm.

FIG. 17 is a bar graph of the Percentage of Ki67 Positive Basal Keratinocytes in Bioengineered Skin Substitutes. The number of basal keratinocytes that were Ki67 positive cells were counted in each channel as well as total number of basal keratinocytes and the percentage positive was determined. For native tissues, basal keratinocytes that were Ki67 positive as well as total basal keratinocytes were counted over a length ranging from 650 μm to 950 μm based on topographical features. Values are reported as averages +/−SEM. For 50 μm, 100 μm, 200 μm, 400 μm widths, and epithelialized DED at 3 days, n=5, 6, 7, 11, and 4, respectively. For 50 μm, 100 μm, 200 μm, 400 μm, and epithelialized DED at 7 days, n=4, 5, 11, 10, and 4, respectively. Samples for foreskin tissues are n=5. For breast tissue 3 separate sections of the same tissue were evaluated.

FIG. 18 is photographs of Keratinocyte Colonies with β₁ and Nuclear Expression. Keratinocytes after 4 days of co-culture were immunostained for β₁ (red) and nuclei (blue) expression. Images A and B represent phase contrast and merged fluorescent images obtained in control wells, respectively. Scale bar=100 μm. Images C and D represent phase contrast and merged fluorescent images, respectively captured at 10× to evaluate cells and expression in total colonies. Scale bar=100 μm. Images E and F represent phase contrast and merged fluorescent images captured at 40× to demonstrate perinuclear expression of β₁. Scale bar=5 μm.

FIG. 19 is photographs of Beta-1 Expression of Basal Keratinocytes in Bioengineered Skin Substitutes. To determine the localization of β₁ bright basal keratinocytes, immunohistochemistry coupled with digital image analyses was used. FIGS. 19A, 19D, 19G, 19J, 19M, and 19P are images with β₁ expression in red and FIGS. 19B, 19E, 19H, 19N, and 19Q are images with β₁ expression in red and nuclear expression in blue. FIGS. 19C, 19F, 19I, 19M, and 19R are plots of the average relative fluorescence intensities (RFI) of cell-cell borders in the region evaluated. Dashed lines in 19C, 19F, and 19I separate flat regions from the channel. It can be seen that for the 100, 400, and DED samples (FIGS. 19B, 19E, 19H, and 19N) β₁ bright cells localized to the rete ridges, whereas in native foreskin β₁ bright cells localized to the tips of the dermal papillae. Additionally when evaluating the flat region of the bioengineered skin substitute, β₁ cells were heterogeneously distributed. Each cell was measured and the average RFI was reported. Insert in A and B represent controls for β₁ and β₁ and nuclear staining. Error bars represent 100 μm in all images.

### DETAILED DESCRIPTION OF THE INVENTION

It was determined that the extracellular matrix protein fibronectin (FN) found in the cellular microenvironment of the DEJ enhanced keratinocyte attachment, proliferation, and epithelialization of a collagen based basal lamina analog. It was also found that the collagen material used to create the basal lamina analog as well as the FN conjugation strategy to this material significantly influenced the bioactivity of FN and its ability to modulate keratinocyte functions through integrin based mechanism. To investigate spatial tissue organization and the role it plays in the cellular microenvironment of the DEJ on epithelialization and epidermal stem cell localization, photolithography coupled with materials processing techniques was used to create microfabricated basal lamina analogs. It was determined that epidermal thicknesses found in narrow channels of microfabricated basal lamina analogs (50 μm and 100 μm widths with 200 μm depths) were similar to cultures on de-epithelialized acellular dermis and native foreskin tissues after 7 days of in vitro culture. It was also determined that the microfabricated basal lamina analogs created an epidermal stem cell niche that promoted epidermal stem cell clustering in the channels which is critical for longevity of the tissue.

A platform technology was developed that was specifically used to produce a highly functional bioengineered skin substitute with regenerative capacity that mimics native skin. Through the use of this technology, further improved bioengineered skin substitutes can be made by incorporating epidermal structures of native skin including hair follicles and sweat glands as well as improve overall cosmetic appearance. Additionally, this bioengineered skin substitute can serve as a model system to further the understanding of pathological conditions and diseases of the skin as well as facilitate robust preclinical screenings of epidermal responses to new therapeutic agents as well as to cosmetic and chemical products.

Carbodiimide Conjugation of Fibronectin on Collagen Basal Lamina Analogs Enhances Cellular Binding Domains & Epithelialization

Strategically modify a biomaterial surface to increase the availability of the central cellular binding domain of fibronectin, which has been shown to promote attachment and subsequent intracellular signaling events, is useful for enhancing epithelialization of bioengineered skin substitutes as well for engineering other functional tissues. The current invention is directed, in part, to evaluating the presence of the central cellular binding domain of FN on collagen membranes and to analyze how the presentation of this binding site effects epithelialization. Using an in vitro skin model, keratinocyte and overall graft morphology, epidermal thickness, and proliferation were evaluated on the surface of collagen-GAG membranes. Fibronectin was found to promote epithelial layers on dermal scaffolds that were found to be morphologically similar to that of native skin. When evaluating proliferation in this model system, it was found that FN treated surfaces enhanced the number of proliferative cells at 3 days of air/liquid (A/L) interface culture. To correlate these findings with the presentation of FN on the surfaces, the availability of the central cellular binding domain on collagen-GAG membranes was evaluated. Self-assembled collagen membranes, fabricated from soluble type I collagen molecules (CI) were developed to further enhance the presentation of FN on the surfaces of basal lamina analogs. The performance of the self-assembled collagen membranes was compared to collagen-GAG membranes. The invention also relates to a method of covalently modifying the surfaces of self-assembled CI membranes with FN using a carbodiimide conjugation strategy, specifically (1-ethyl-3-(3-dimethylaminopropyl)carbodiimide hydrochloride (EDC). Finally, the effect of EDC conjugation on the presentation and bioactivity of FN was evaluated. Overall, it was demonstrated that the EDC conjugation strategy greatly enhances the availability of the central cellular binding domain of FN. This modification strategy also can be used to increase the rate of epithelialization on the surfaces of basal lamina analogs.

FN Enhances Epithelialization of Keratinocytes on Basal Lamina Analogs

Graft Morphology and Epidermal Layer Thickness on Collagen-GAG Basal Lamina Analogs

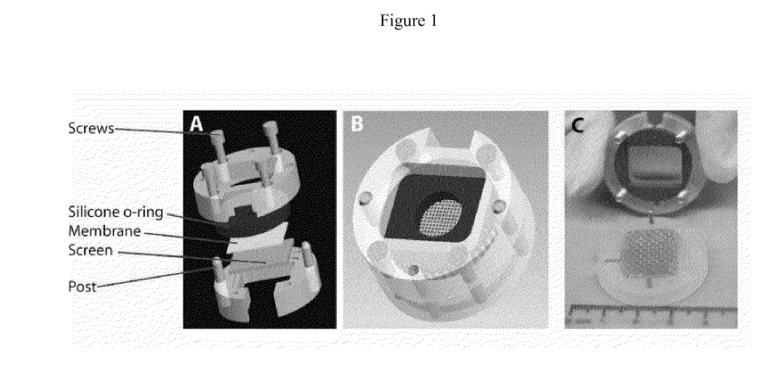

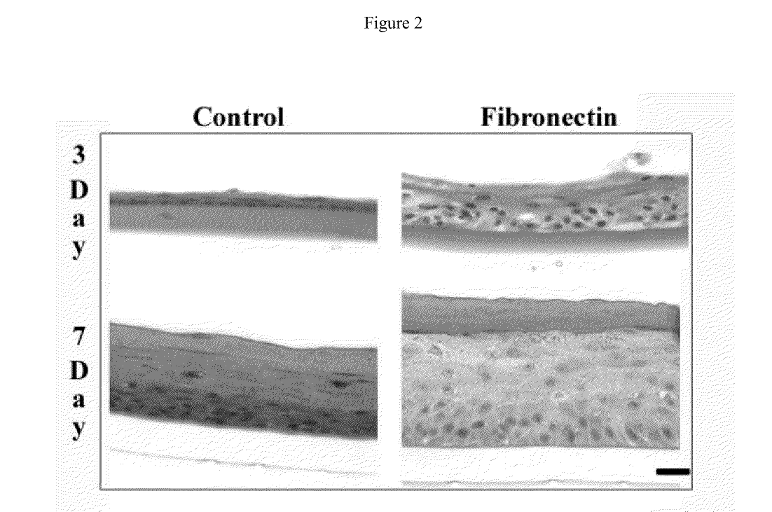

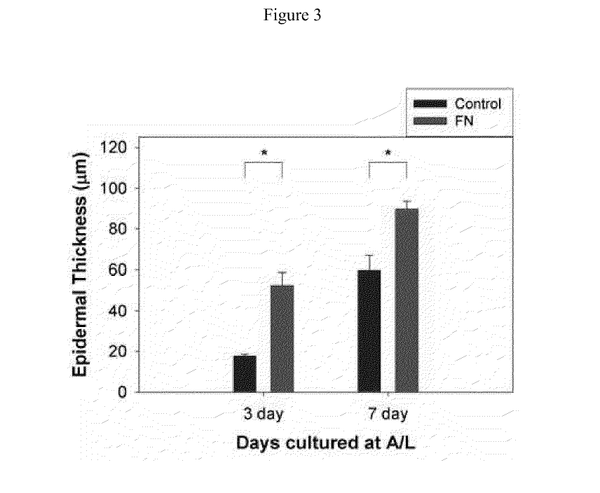

The effect of passively adsorbed FN on graft morphology and epithelial layer thickness of keratinocytes was evaluated using custom built A/L interface culture devices (FIG. 1). Fibronectin (100 μg/ml) was passively adsorbed to the surfaces of collagen-GAG membranes and keratinocytes were cultured on these basal lamina analogs for 3 or 7 days at the A/L interface. FIG. 2 shows histological results of control grafts compared with grafts that were passively adsorbed with FN, cultured for 3 or 7 days and stained with H&E. A thicker epidermal layer formed on membranes modified with FN when compared with control membranes at 3 or 7 days of culture at the A/L interface. At 3 days, 17.7+/−0.9 and 52.4+/−6.3 μm were found for control and FN treated membranes, respectively and at 7 days, 59.6+/−7.4 and 89.6+/−4.2 μm were found for control and FN treated membranes, respectively. These differences in epithelial thickness between control and FN treated surfaces were statistically different at both time points (FIG. 3).

Keratinocyte Proliferation on Collagen-GAG Basal Lamina Analogs

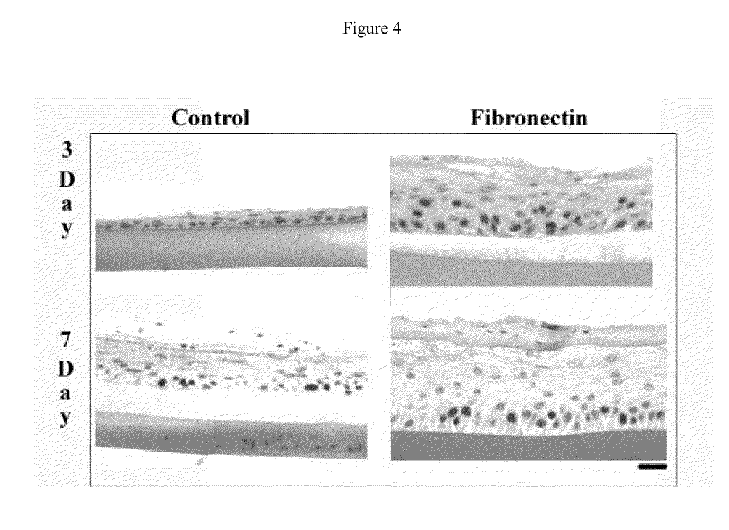

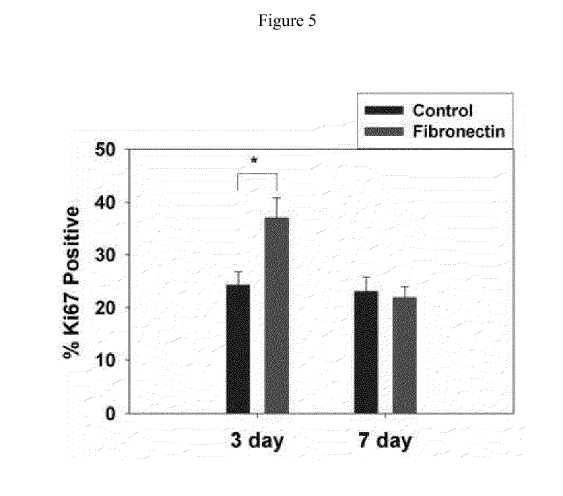

To analyze keratinocyte proliferation, the presence of Ki67 in basal keratinocytes was measured on the surfaces of cultured basal lamina analogs. This protein is present during active phases of the cell cycle and absent from resting cells. FIG. 4 shows histological images of control collagen-GAG membranes and membranes that were passively adsorbed with FN, cultured for 3 or 7 days at the A/L interface and immunoassayed for Ki67. Quantitative analyses of these images are depicted in FIG. 5. At 3 days of culture, positive Ki67 basal keratinocytes were counted and control surfaces and FN treated surfaces had 24.3+/−2.5% and 37+/−3.9% Ki67 positive basal cells, respectively and at 7 days control surfaces and FN treated surfaces had 23+/−2.7% and 21.9+/−2.1% Ki67 positive basal cells, respectively. The percentage of basal keratinocytes expressing Ki67 on FN modified membranes was statistically different than on control membranes at 3 days of culture, however at 7 days of culture no differences were detected.

Availability of Cellular Binding Domain of FN Corresponds to Keratinocyte Attachment on Collagen-GAG Basal Lamina Analogs

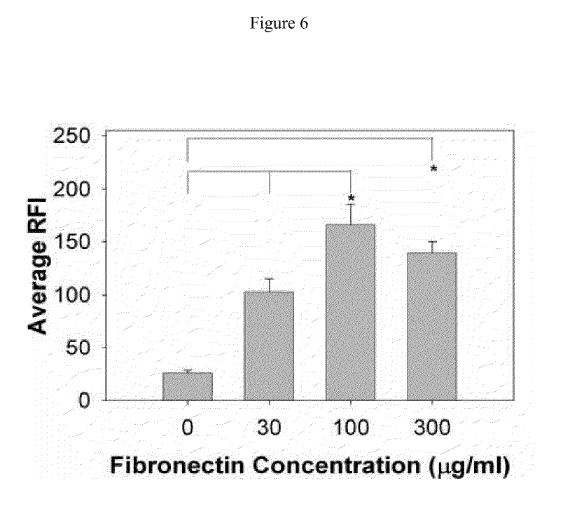

The availability of the cellular binding domain of FN, specifically the domain that encompasses both the RGD and PHSRN binding sequences, was analyzed on the surfaces of collagen-GAG basal lamina analogs using an antibody directed towards this site. Relative fluorescence intensity (RFI) measurements were made on several regions of interest and an average value was reported. When FN was passively adsorbed to collagen-GAG membranes at 30, 100, or 300 μg/ml cellular binding sites plateaued at a concentration of 100 μg/ml (FIG. 6). The fluorescence intensity values obtained at 100 μg/ml and 300 μg/ml were statistically greater than those at 30 μg/ml of FN. This data directly corresponds with keratinocyte attachment measurements made on FN treated collagen-GAG membranes in a previously published study.

EDC Conjugation of FN on Self-Assembled CI Basal Lamina Analogs Promotes Increased Cellular Binding Site Availability

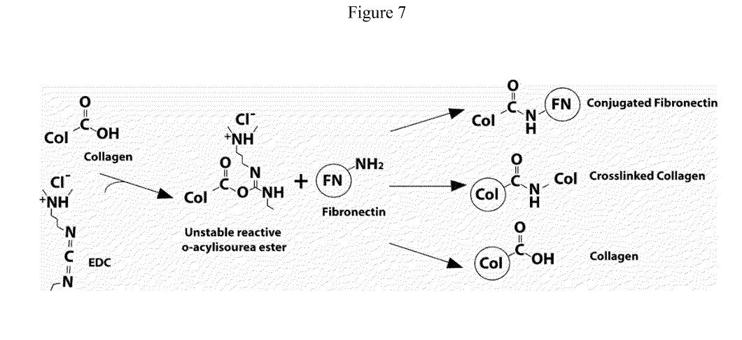

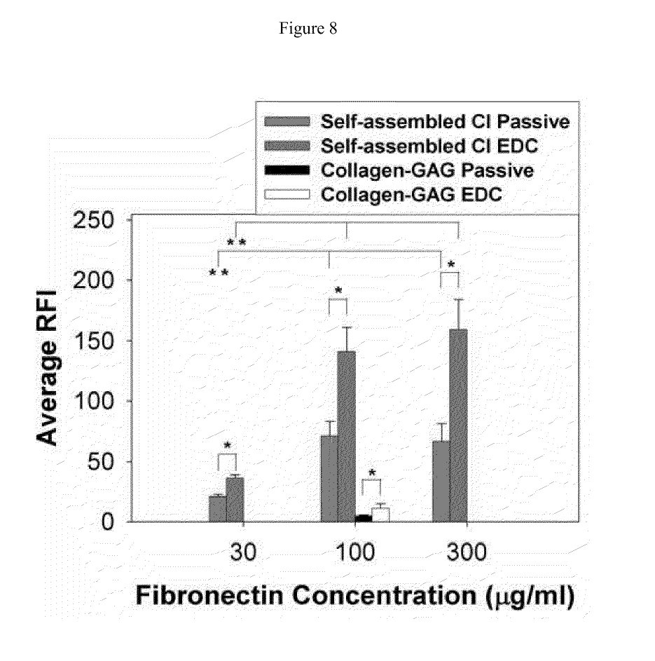

The effects of covalently binding FN to the surface using an EDC conjugation strategy were analyzed to determine whether the presentation of FN cellular binding sites on the surfaces of collagen-GAG basal lamina analogs could be increased (FIG. 7). Since it was found that 100 μg/ml of FN saturated the surfaces of the collagen-GAG membranes, this concentration was chosen for evaluation using both adsorption and EDC modification strategies. When analyzing collagen-GAG membranes, the difference between passive adsorption and EDC conjugation of 100 μg/ml of FN, was that EDC conjugation statistically increased the average RFI on the surfaces of FN treated membranes, suggesting that these membranes have a greater capacity for cellular binding (FIG. 8).

The availability of cellular binding domains on the surfaces of self-assembled CI basal lamina analogs was evaluated for both passive adsorption of FN and EDC conjugation of FN. These findings were compared with both passive adsorption and EDC conjugation of FN on collagen-GAG collagen basal lamina analogs (FIG. 8). When the passive adsorption of 100 μg/ml of FN on collagen-GAG was compared to that on self-assembled CI membranes, a significant increase in average RFI was observed on the self-assembled CI membranes. Similarly, when the binding efficiency of FN using an EDC conjugation strategy at a concentration of 100 μg/ml of FN on collagen-GAG was compared to that on self-assembled CI membranes, a significant increase was found in average RFI on the self-assembled CI membranes. Additional concentrations were analyzed, for both passive adsorption and EDC conjugation of FN on self-assembled CI membranes, to evaluate whether saturation levels of RFI were obtained. When evaluating lower and higher concentrations of FN (30 and 300 μg/ml, respectively), there were no statistical differences between FN concentrations of 100 μg/ml and 300 μg/ml and the 100 μg/ml concentration had statistically increased values over the 30 μg/ml concentration, regardless of the conjugation strategy that was used. This data indicates that the presentation of cellular binding domains on the surfaces of self-assembled CI membranes saturated at a FN concentration of 100 μg/ml is similar to the results obtained for collagen-GAG membranes.

EDC Conjugation of FN on Self-Assembled CI Basal Lamina Analogs Enhances Epidermal Layer Thickness

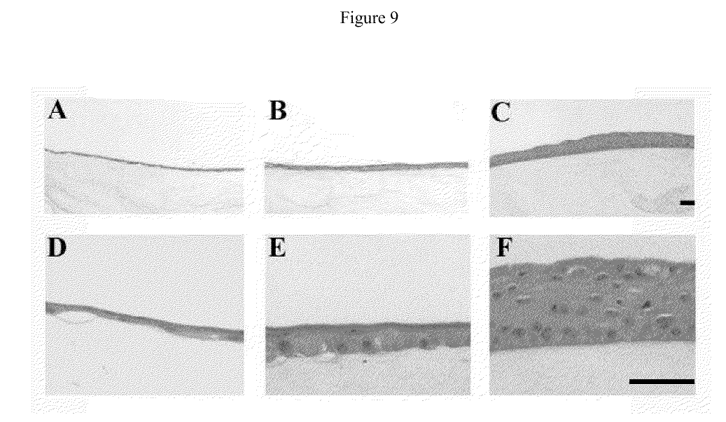

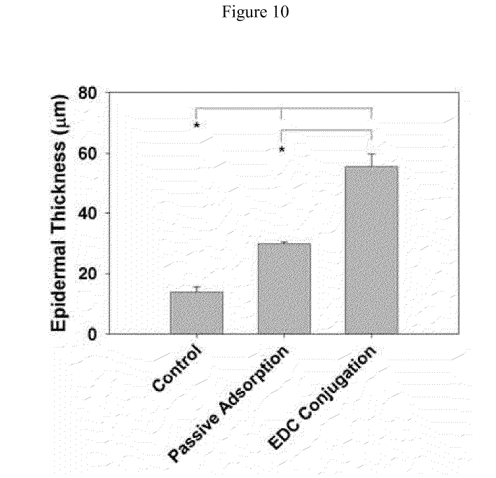

Fibronectin was covalently bound to the surface of self-assembled CI membranes using EDC and keratinocytes were cultured on the surface of basal lamina analog for 3 days at the A/L interface to determine whether increased cellular binding sites on the new model system promoted an increase in epithelial layer thickness. FIG. 9 shows a typical image of a cultured epithelial layer on an untreated self-assembled CI membrane (9A and 9D), a basal lamina analog with FN passively adsorbed to the surface (9B and 9E), and a basal lamina analog with FN that was EDC conjugated to the surface (9C and 9F). These images suggest that the thickness of the epidermal layer on the scaffold corresponds to the presentation and availability of FN central cellular binding domains. Morphometric analyses of these epidermal layers (FIG. 10) showed that there were statistical differences between all surfaces analyzed.

Microenvironments of the Basal Lamina Influence Epithelialization and Stem Cell Localization on Bioengineered Skin Substitutes

Understanding how the microenvironment found at the DEJ influences ESC localization and promotes the development of a functional tissue is critical in the development of the next generation of bioengineered skin grafts as well as for longevity of the tissue. Incorporating microfabricated basal lamina analog, containing biochemical and microtopographic features mimicking those found at the DEJ, can promote enhanced epithelialization and epidermal stem cell clustering on the surface of novel dermal scaffolds. Previously, basal lamina analogs were created on the surface of collagen-glycosaminoglycan (GAG) dermal scaffolds that recapitulate the native topographical features found at the DEJ utilizing photolithography and material processing techniques. It was determined that topographical features with the greatest aspect ratios enhanced keratinocyte stratification, proliferation, and differentiation. Additionally, it was found that passively adsorbing the ECM protein FN, on the surface of flat collagen-GAG membranes increased keratinocyte attachment over non-modified control collagen-GAG surfaces by 22%. When further investigating FN binding domains and conjugation strategies, it was determined that carbodiimide conjugation, could enhance the presentation of the cellular binding site domain of FN on the surfaces of self-assembled CI membranes. This invention includes a novel system that incorporates both microtopographic and biochemical features to enhance epithelialization. Histological stains and immunohistochemistry coupled with quantitative morphometric analyses of microscopic images were used to determine the effect of this combined microenvironment on epithelial thickness, morphology, proliferation, and ESC localization. Furthermore, the bioengineered skin substitutes that contain microfabricated basal lamina analogs were compared with both native adult and neonatal tissues as well as de-epithelialized acellular dermis (DED) cultured under the same conditions. Overall, the present invention is a bioengineered skin substitute with a microfabricated basal lamina analog that imparts the ability to direct ESC localization, as well as a model system to further investigate advanced ESC markers and the mechanisms by which ESC localization occurs.

Development of Bioengineered Skin Substitutes Containing Microfabricated Basal Lamina Analogs

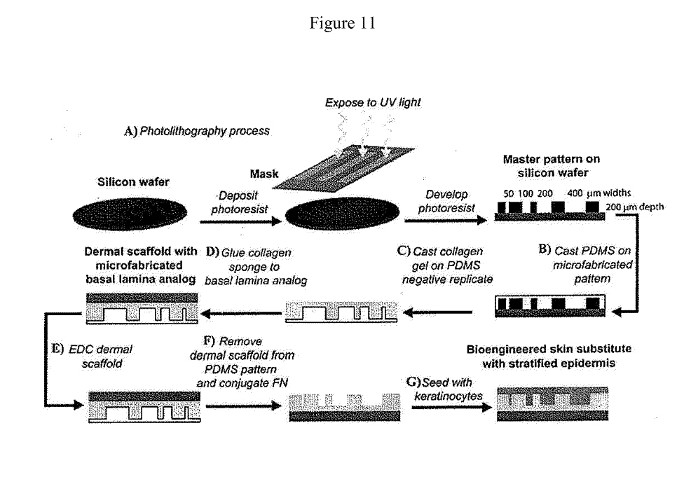

In native skin, the DEJ is not flat, but rather has a microtopography that conforms to a series of ridges and invaginations that, in combination with the biochemical composition, provides a microenvironment to direct basal keratinocyte functions. To investigate the role of this microenvironment on epithelialization and the regenerative capacity of bioengineered skin substitutes, a process was developed to create a dermal scaffold containing microfabricated basal lamina analogs composed of a defined starting collagen material EDC conjugated with FN (FIG. 11).

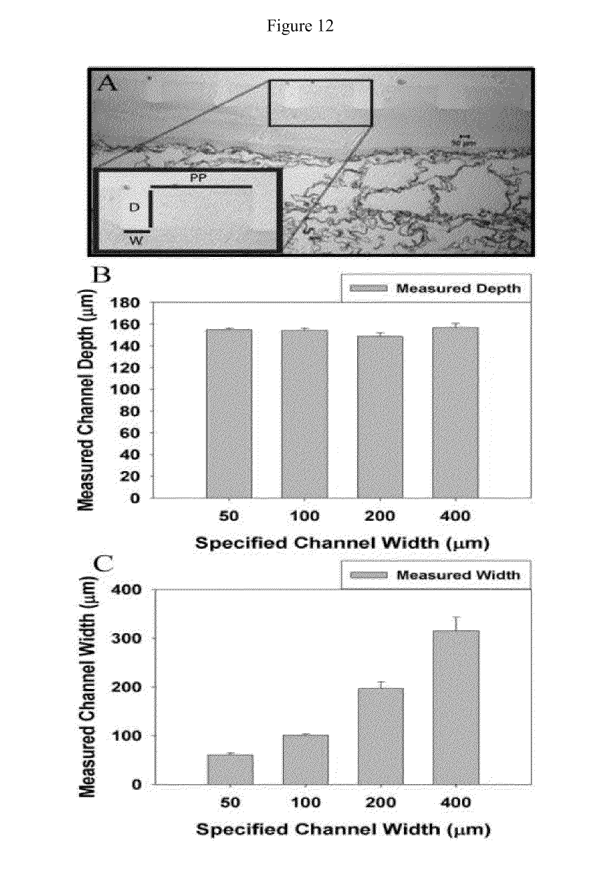

Photolithography was utilized to create a master pattern containing channels with specified depths of 200 μm and widths of 50, 100, 200, and 400 μm. Type I collagen was cast onto a PDMS negative replicate of the master pattern and allowed to polymerize. A collagen-GAG sponge was then adhered to the back of the microfabricated self-assembled type I collagen membrane and the composite was EDC crosslinked to provide mechanical and degradation stability, as well as to provide sites for chemical conjugation of FN to the topographical features. The topographical features provided on the surface of the basal lamina analog were analyzed through histological techniques before cellular seeding. Depths and widths of the channels were measured using Image J (FIG. 12). It was found that the depths for each channel were approximately 150 μm (FIG. 12B and Table 1) and widths for 50 μm were 60.8+/−3.8, 100 μm were 101.2+/−2.4, 197.1+/−13.5, and for 400 μm 315.7+/−27.9 (FIG. 12C and Table 1).

TABLE 1
Specified and Measured Topographical Features of Basal Lamina Analog

| Specified Values | | | | Analyzed |
|---|---|---|---|---|
| Width | Depth | Measured Values | | Channels |
| (μm) | (μm) | Width (μm) | Depth (μm) | Width (μm) |
| 50 | 200 | 60.8 +/− 3.8 (4) | 154.9 +/− 1.4 (4) | 53-68 |
| 100 | 200 | 101.2 +/− 2.4 (5) | 154.3 +/− 2.1 (5) | 96-106 |
| 200 | 200 | 197.1 +/− 13.5 (5) | 148.8 +/− 3.4 (5) | 170-224 |
| 400 | 200 | 315.7 +/− 27.9 (5) | 156.9 +/− 3.9 (5) | 260-371 |

Microenvironments Provided By a Microfabricated Basal Lamina Analog Influence Epidermal Thickness and Morphology of the Epidermal Layer of Bioengineered Skin Substitutes

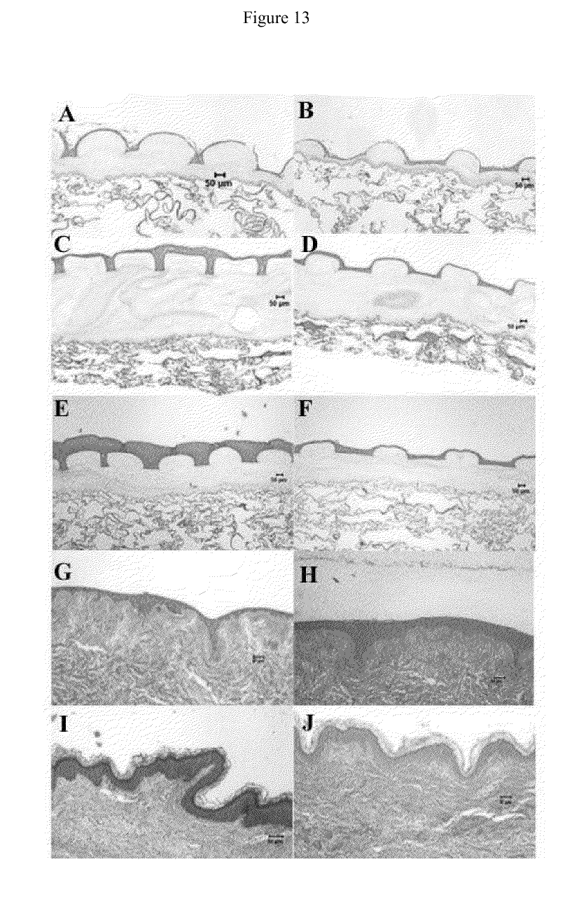

The effect of the microenvironment on epidermal thickness was analyzed at 3 or 7 days of A/L interface culture on a bioengineered skin substitute containing a microfabricated basal lamina. Epidermal thickness was evaluated using histological techniques and quantitative morphometric analyses of microscopy images. FIG. 13 displays representative hematoxylin and eosin stained channels that were evaluated. Previously it was shown that the presence of FN conjugated to the surface of a self-assembled CI basal lamina analog enhances epithelialization. When comparing basal lamina analog surfaces without FN conjugation (FIGS. 13A and 13B) with basal lamina analog surface with FN conjugation (FIGS. 13C and 13D), it can be seen that the FN surfaces have a continuous multi-layer of cells, regardless of topographical geometry (FIG. 13A-13D) in comparison with the non-continuous multi-layers of cells found cultured on the surfaces without FN.

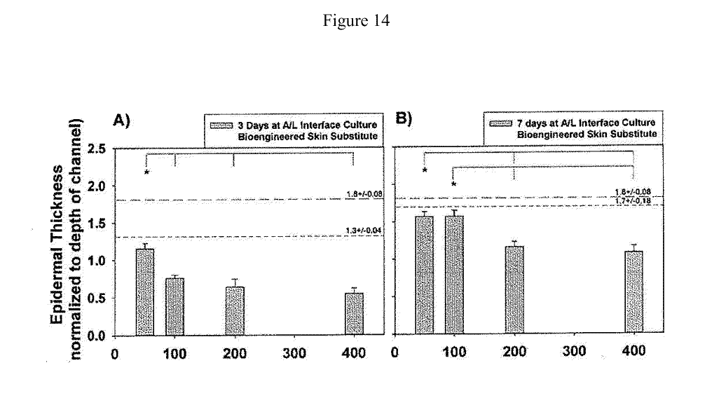

When comparing grafts cultured with FN at various time points, it can be seen that the geometrical features play a role in epidermal thickness. At 3 days of A/L interface culture, channels with widths of 50 μm have a noticeably thicker epidermis than channels with widths of 200 μm (FIGS. 13C and 13D, respectively). Epidermal thickness normalized to the depth of the channel at 3 days of A/L interface culture for the 50 μm channels, was statistically greater than the thickness measured for the 100 μm width, 200 μm width, and 400 μm width channels (FIG. 14A)

The epidermal layer on the bioengineered skin substitutes cultured in the 50 μm width channels was similar in thickness and morphology to the epidermal layer cultured on DED for 3 days at the A/L interface (FIG. 13G). When quantifying the epidermal thickness, no statistical differences were found between the decellularized dermis and the 50 μm width channels at 3 days (FIG. 14A). At the 7 day A/L interface culture time point for bioengineered skin substitutes, the 50 μm width and 100 μm width (FIG. 13E) channels have similar morphologies and epidermal thicknesses and when compared to the 200 μm width channels (FIG. 13F) are much thicker.

Epidermal thickness for the 50 μm width and 100 μm width channels had similar values, and were both statistically different from the 200 μm width and 400 μm width channels (FIG. 14B). When comparing the bioengineered skin substitutes at 7 days of A/L interface culture to the epidermal layer on DED (FIG. 13H) and native skin (FIGS. 13I and 13J), it can be seen that the 50 μm width and 100 μm width channels have similar morphologies and thickness. No statistical differences were found in epidermal thickness between 50 μm width and 100 μm width channels. Additionally, no statistical differences were found in epidermal thickness between 50 μm width and 100 μm width channels and the epidermal thickness of cells cultured for 7 days at A/L interface on DED or foreskin tissue (FIG. 14B).

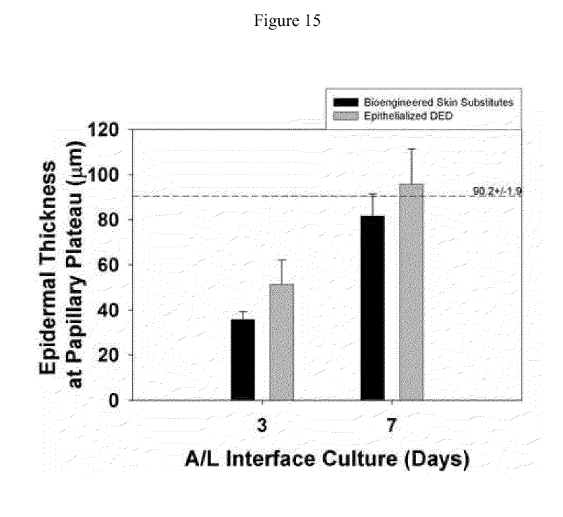

The epidermal thicknesses at the papillary plateaus for the bioengineered skin substitutes were measured to compare the thicknesses achieved regardless of depth of channels or depths of rete ridges. (FIG. 15 see FIG. 12A for papillary plateau measurement clarification if necessary). The papillary plateaus between all channels were then averaged and compared to the epidermal thicknesses on the papillary projections for epithelialized DED and foreskin tissue. At 3 days of A/L interface culture, bioengineered skin substitutes and epithelialized DED were not statistically different from each other but different from native foreskin. At 7 days of A/L interface culture, the epidermal thicknesses at the papillary plateau were not statistically different between any measured tissues.

Proliferation Capacity of Bioengineered Skin Substitutes is Affected by the Microenvironment Provided by a Microfabricated Basal Lamina Analog

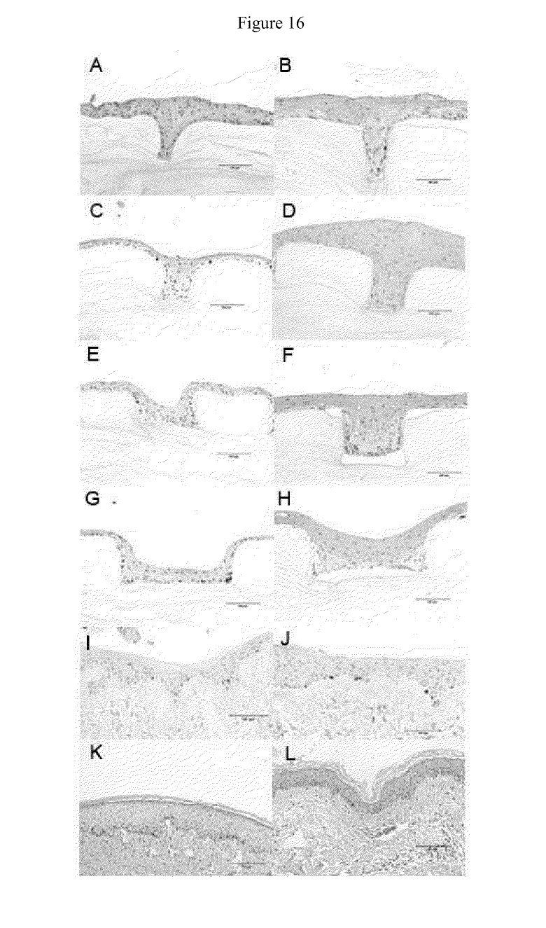

To determine the effects of microtopography on cell proliferation bioengineered skin substitutes and epithelialized DED were evaluated after 3 or 7 days of A/L interface culture. The samples were stained for the nuclear proliferation antigen Ki67 and counterstained with hematoxylin (FIG. 16).

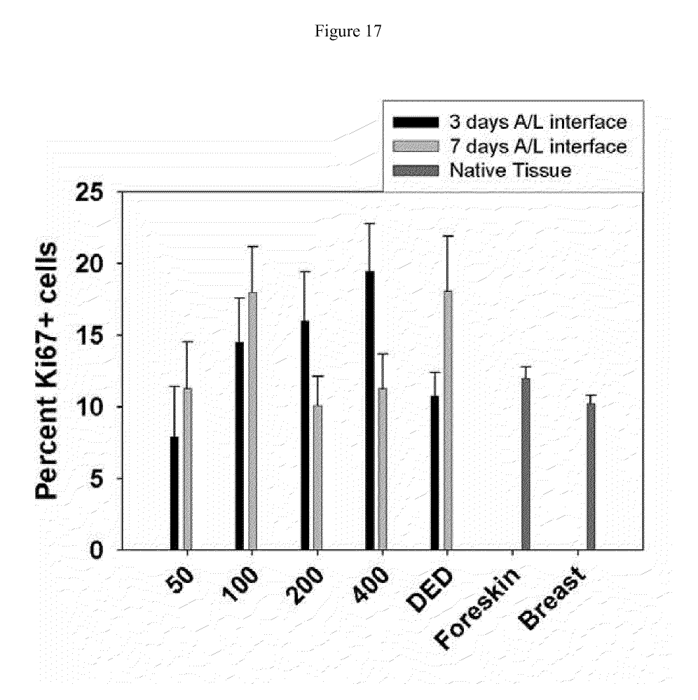

Foreskin and breast tissues were also evaluated as native skin controls (FIG. 16K and L). The percentage of Ki67 positive cells was quantified in each channel, or over the entire basal lamina for epithelialized DED or native tissues (FIG. 17).

At 3 days of A/L interface culture, the 50 μm width channels had the lowest average percentage of Ki67 positive cells (approximately 7.5% FIG. 17), and 40% of these channels had zero positive cells. At 7 days of A/L interface culture, the 50 μm width channels had a slightly higher average percentage of Ki67 positive cells than at 3 days, and 20% of the channels analyzed had zero positive cells (FIG. 17). When analyzing the 100 μm width channels after 3 days of A/L interface culture, it was found that all channels contained positive cells and an average of approximately 15% Ki67 positive cells was found. At 7 days of A/L interface culture, the percentage of Ki67 positive cells was approximately the same as at 3 days and all 100 μm width channels analyzed contained positive cells (FIG. 17).

The 200 μm width and 400 μm width channels had similar values and trends at both 3 and 7 days of A/L interface culture. At 3 days of A/L interface culture the 200 μm width channels had approximately 15% Ki67 positive cells and the 400 μm width channels had approximately 18% Ki67 positive cells. At 7 days, both channels decreased in percentage Ki67 positive cells to approximately 10% (FIG. 17). Epithelialized DED exhibited approximately 10% Ki67 positive cells at 3 days of A/L interface culture and approximately 18% Ki67 positive cells at 7 days of A/L interface culture. When analyzing native tissues, it was found that the basal keratinocytes of neonatal foreskin were approximately 12% Ki67 positive and basal keratinocytes in breast tissue were approximately 10% Ki67 positive. Overall our Ki67 data suggests that no significant differences exist among any sample evaluated at either 3 days or 7 days of A/L interface culture (FIG. 17) When performing a power analysis, it was found that P<0.8 for both the 3 and 7 day data, therefore to further support these findings, sample sizes need to be increased.

Beta-1 Expression in Keratinocyte Colonies Detected in Edge Keratinocytes

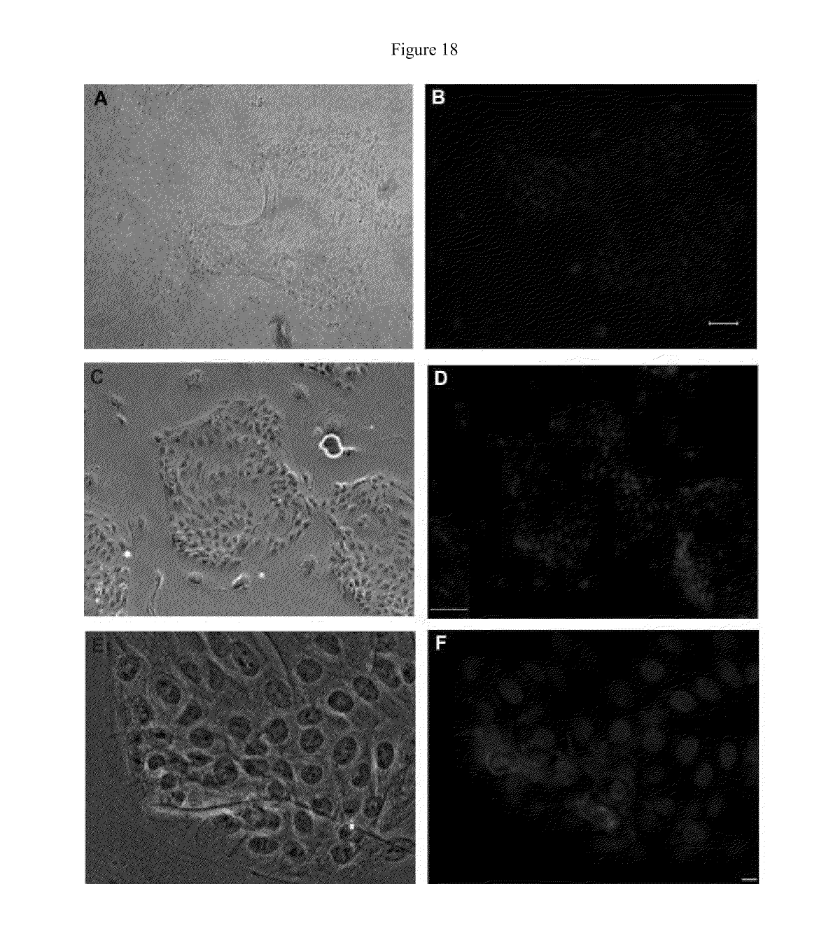

The expression of β₁ in colonies of keratinocytes was evaluated after 4 days of co-culture with a feeder layer of J2s. It was found that for all colonies in each culture well, β₁ expression was found in the periphery of keratinocytes on the perimeter of each colony. To analyze the localization of β₁ bright regions, the maximal fluorescence intensity was determined so that no saturation occurred in the image. This value was then divided into three equal regions, thus giving three regions of integrin expression values (bright, medium, and dull). Any value in the top third was considered β₁-bright similar to previously reported literature. When analyzing the percentage of cells were β₁-bright it was determined that 25% +/−0.1 of the colony were β₁-bright. FIG. 18A and 18C are phase contrast images that display a representative colony at 10 and 40× and 18B and 18D are fluorescent images displaying β₁ expression (red) and cell nuclei (blue) at 10 and 40×, respectively.

Microenvironments Control Spatial Localization of β1-Bright Basal Keratinocytes

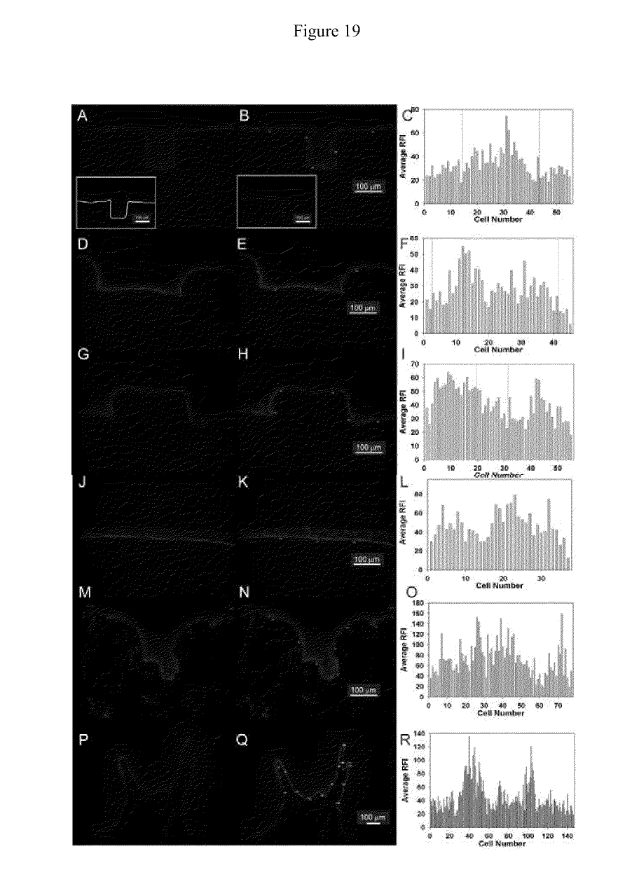

Immunohistochemistry coupled with quantitative digital image analyses was utilized to determine localization of β₁-bright keratinocytes in bioengineered skin substitutes, epithelialized DEDs, and in native foreskins. Fluorescent intensity values were determined for cell-cell borders similar to previously reported literature for 3 day A/L interface cultures. FIG. 19 displays representative images of 100 μm width channels (FIGS. 19A and 19B), 400 μm width channels (FIGS. 19D, 19E, 19G, and 19H), flat regions of bioengineered skin substitutes (FIGS. 19J and 19K), epithelialized DED (FIGS. 19M and 19N), and neonatal foreskin (FIGS. 19P and 19Q).

It was found that in the 100 μm width (FIGS. 19A, 19B, and 19C) and 400 μm width (FIGS. 19D, 19E, and 19F) channels, there were no β₁-bright cells in the flat sections next to the channels (papillary plateaus), but in the channels 16.7% and 23% of basal keratinocytes were β₁-bright, respectively (dashed lines in 19C and 19F separate flat regions from channel regions). FIGS. 19G, 19H, and 19I are another representative image of the 400 μm width channels demonstrating β₁-bright regions localized to the corners of the channels. In the corners of the 400 μm width channels we found that 50% of the total basal keratinocyte population was β₁-bright. When β₁ was evaluated on flat regions of the bioengineered skin substitutes, 30% of the basal keratinocyte population was β1-bright, however the cells were not localized, but heterogeneously distributed (FIGS. 19J, 19K, 19L). The expression of β₁-bright basal keratinocytes on epithelialized DED was found to be 15.6% and the β₁-bright cells were localized to the rete ridges. Additionally, β₁ expression was evaluated in native foreskin tissue. It was found that the β₁-bright basal keratinocytes localized to the tips of the papillary projections. Overall 6.8% of the total basal keratinocyte population was β₁-bright.

### EXAMPLES

A/L Interface Culture Devices

To evaluate the effect of FN on epithelialization of bioengineered skin substitutes, a custom designed device was developed to analyze membranes which are precisely conjugated with FN and cultured at the A/L interface. This system creates an individual well on the surface of a collagen membrane and allows for a tight seal to be made on the surface of the composite assuring that FN placement is in the center (FIG. 1).

Basal Lamina Analog Production

Collagen-GAG Membranes

A collagen-GAG dispersion containing type I collagen (5 mg/ml) and GAG (0.18 mg/ml) was prepared by placing lyophilized bovine hide derived collagen (Semed-S, Kensey Nash Corp., Exton, Pa.) in acetic acid (EMD Chemicals, Inc., Gibbstown, N.J.) and homogenizing (20,000 rpm) at 4° C. for 90 minutes resulting in a bovine-derived collagen suspension.

Shark cartilage chondroitin 6-sulfate (Sigma, St. Louis, Mich.) was dripped into the blending collagen dispersion and blended for an additional 90 minutes. Once fully blended, the collagen-GAG suspension was degassed by centrifugation. To produce membranes, the suspension was cast onto flat polydimethylsiloxane silicone elastomer (PDMS, Sylgard 184, Dow Corning Corp., Midland, Mich.) molds 9.62 cm2 in area, and allowed to air dry in a laminar flow hood at room temperature. The membrane was then gently peeled from the PDMS surface and dehydrothermally (DHT) crosslinked according to previously published methods for 24 hours 9 Membranes were then stored in a desiccator until use.

Self-Assembled Type I Collagen Membranes

Acid-soluble type I collagen (CI) was extracted from rat tail tendons using protocols previously described. Rat tails were received from animals that were euthanized for other protocols, which were approved by Worcester Polytechnic Institute, Worcester, Mass., Institutional Animal Care and Use Committee. Briefly, rat tail tendons were extracted from the tails of 13 Sprague Dawley rats, rinsed in dPBS (Hyclone, Logan, Utah), and dissolved in 1600 ml of 3% acetic acid at 4° C. overnight. The resulting solution was centrifuged at 8590 rpm for 2 hours and 320 ml of a 30% NaCl (Sigma) solution was dripped into the supernatant at 4° C. The resulting solution was allowed to sit for at least 1 hour at 4° C. without disruption and then centrifuged at 4690 rpm for 30 minutes to separate precipitated and liquid material. The precipitated material was resuspended in 400 ml of 0.6% acetic acid and dialyzed for 4 hours against 1 mM HCl (J T Baker, Phillipsburg, N.J.) and the dialysis solution was changed every 4 hours until a clear collagen solution was obtained. This solution was lyophilized and stored in a sealed container at 4° C., until use. Lyophilized collagen was dissolved in 5 mM HCl to obtain a working solution of 10 mg/ml. To produce self-assembled CI membranes, 800 μl of the soluble CI solution was neutralized using 200 μl of 5× Dulbecco's Modified Eagle's Medium (DMEM, Invitrogen, Carlsbad, Calif.) with 0.22 M NaHCO3 and 40 μl of 0.1 M NaOH (Sigma) at 37° C. for 18 hours on circular PDMS molds.

### FN Surface Modification of Collagen Membranes

Passive Adsorption of FN to Collagen Membranes

Fibronectin (BD Biosciences, Bedford, Mass.) was resuspended according to manufacturer's recommendations in 1 ml of dH2O and diluted to desired concentrations (30, 100, and 300 μg/ml) using dPBS. For in vitro culture on basal lamina analogs, all collagen membranes were placed in A/L culture devices (FIG. 1) and FN (100 μg/ml) was placed in the well created on the surface of the collagen membrane and allowed to adsorb overnight at room temperature. For FN cellular binding site evaluation of basal lamina analogs, collagen membranes were placed in a custom high throughput screening device and FN was placed into each individual wells at 30, 100, and 300 μg/ml for self-assembled CI membranes, and at 100 μg/ml for collagen-GAG membranes overnight at room temperature.

1-ethyl-3-(3-dimethylaminopropyl)carbodiimide hydrochloride (EDC) Conjugation of FN to Collagen Membranes

Using protocols previously described to crosslink collagenous materials, the molar ratio of 5:1 (EDC to carboxylic acid groups in collagen) was used to conjugate FN to the surfaces of collagen-GAG and self-assembled CI membranes. The theoretical amount of collagen used for calculations assumed that 1 g of type I collagen contained 1.2 mmol COOH. Collagen-GAG membranes contained 12.5 mg of type I collagen and self-assembled CI membranes contained 8 mg of type I collagen, thus receiving 0.075 mmol EDC and 0.048 mmol EDC, respectively. 1-ethyl-3-(3-dimethylaminopropyl)carbodiimide hydrochloride (Sigma) was dissolved in 50 mM MES hydrate (Sigma) dissolved in 40% ethanol (Pharmco Products, Inc., Brookfield, Conn.) at a pH 5.5 and 1.25 ml of solution was placed on collagen-GAG membranes and 0.8 ml was placed on self-assembled CI membranes for 4 hours. For in vitro culture on basal lamina analogs, the membranes were removed from the EDC solution and immediately placed into the A/L culture devices and 100 μg/ml of FN was placed in the well created on the surface of the collagen membrane over night at room temperature. For FN cellular binding site evaluation, the membranes were immediately placed in a custom high throughput screening device and FN was placed into each individual wells at 30, 100, and 300 μg/ml for self-assembled CI membranes, and at 100 μg/ml for collagen-GAG membranes overnight at room temperature.

Culture of Neonatal Human Keratinocytes

Neonatal keratinocytes were cultured as previously described. Neonatal foreskins were obtained from non-identifiable discarded tissues from UMass Memorial Medical Center, Worcester, Mass. and were approved with exempt status from the New England Institutional Review Board. Keratinocyte isolations were performed using an enzymatic treatment with a dispase (Gibco, Gaithersburg, Md.) solution. The cells were propagated on a feeder layer of 3T3-J2 mouse fibroblasts (generously donated by Dr. Stelios Andreadis, State University of New York at Buffalo, Buffalo, N.Y.) and cultured according to methods previously described using keratinocyte media consisting of a 3:1 mixture of DMEM (high glucose) and Ham's F-12 medium (Invitrogen) supplemented with 10% fetal bovine serum (FBS, Hyclone), 10-10 M cholera toxin (Vibrio Cholerae, Type Inaba 569 B), 5 μg/ml transferrin, 0.4 μg/ml hydrocortisone (Calbiochem, La Jolla, Calif.), 0.13 U/ml insulin, 1.4*10-4 M adenine, 2*10-9 M triiodo-L-thyronine thyronine (Sigma), 1% penicillin/streptomycin (Invitrogen), and 0.01 μg/ml epidermal growth factor (EGF, BD Biosciences). After 5 days of culture, cells were detached using 0.05% Trypsin-EDTA (Invitrogen) and then rinsed with serum free and EGF free keratinocyte media. Passage 2 keratinocytes were used in all experiments.

In vitro Culture of Keratinocytes on Basal Lamina Analogs

After FN adsorption or EDC conjugation of FN to membranes, the membranes were sterilized in composite culture devices using 70% ethanol. Membranes and devices were removed from ethanol and rinsed in sterile dPBS, 3 times for 10 minutes each, and left overnight in sterile dPBS. The composite culture devices were placed into individual wells of a 6-well tissue culture plate and preconditioned for 30 minutes with seeding media consisting of 3:1 mixture of DMEM (high glucose) and Ham's F-12 medium supplemented with 10⁻¹⁰ M cholera toxin, 0.2 μg/mL hydrocortisone (Calbiochem), 5 μg/mL insulin, 50 μg/mL ascorbic acid (Sigma), and 1% penicillin/streptomycin (Invitrogen). Keratinocytes were seeded on the surfaces of the membranes at 500,000 cells/cm² using this media, and allowed to adhere for 2 hours in 10% CO₂ at 37° C. After 2 hours, seeding media containing 1% FBS was placed in each well, completely submerging the grafts. After 24 h, the keratinocyte seeding medium was removed, and the grafts were submerged for an additional 48 h in a keratinocyte priming medium composed of keratinocyte seeding medium (with FBS) supplemented with 24 μM bovine serum albumin (BSA), 1.0 mM L-serine, 10 μM l-carnitine, and a mixture of fatty acids including 25 μM oleic acid, 15 μM linoleic acid, 7 μM arachidonic acid, and 25 μM palmitic acid (Sigma). After 48 h in priming medium, skin equivalents were cultured for 3 or 7 days with an A/L interface medium composed of serum-free keratinocyte priming medium supplemented with 1.0 ng/mL EGF.

Evaluation of Epithelialization

To assess epithelialization on the basal lamina analogs, epidermal thickness and proliferation were evaluated after 3 or 7 days of A/L interface culture. Grafts were fixed in a 10% buffered formalin solution (EMD Chemicals), dehydrated with increasing concentrations of ethanol, cleared with sec-butyl alcohol (EMD Chemicals), and embedded in Paraplast tissue embedding medium (McCormick Scientific, St. Louis, Mo.). Sections of skin equivalents, 6 μm in thickness, were cut in a plane perpendicular to the surface of the epithelial layer using a Leica RM 2235 (Leica Microsystems, Inc, Bannockburn, Ill.). Sections were mounted on poly-L-lysine coated slides (Erie Scientific Company, Portsmouth, N.H.) for hematoxylin and eosin (H&E) staining and mounted on Superfrost Plus slides (VWR, West Chester, Pa.) coated with poly-L-lysine (Sigma) to evaluate proliferation. To evaluate thickness of the epithelial layer, the slides were stained with Harris hematoxylin and eosin (Richard-Allan Scientific, Kalamazoo, Mich.) and then viewed with a Nikon Eclipse E400 microscope (Nikon, Inc., Melville, N.Y.). Images were captured using an RT Color Spot camera (Spot Diagnostics, Sterling Heights, Mich.). Thickness measurements were taken in three areas of the image using Image J software (downloaded from http://rsb.info.nih.gov.ezproxy.umassmed.edu/ij/) and an average value was reported for each graft. For collagen-GAG membranes with and without passive adsorbed FN, at 3 day or 7 day culture, 7 and 4 cultured basal lamina analogs were evaluated, respectively. For self-assembled CI membranes with no treatment, passive adsorption of FN, and EDC conjugation of FN, 3 grafts were evaluated for each condition.

Keratinocyte proliferation was evaluated by detecting the presence of Ki67, a marker for highly mitotic keratinocytes. The tissue sections were deparaffinized in reverse ethanol-xylene washes, and the antigens were unmasked by placing the slides in boiling Vector Unmasking solution (Vector Laboratories, Inc, Burlingame, Calif.) in a Manttra pressure cooker (Manttra, Inc., Virginia Beach, Va.) for 1 minute after maximum pressure was achieved. Slides were then incubated with blocking solution (10% normal horse serum (Vector Laboratories) in dPBS) for 10 min at room temperature and treated with predilute mouse-antihuman Ki67 (Zymed Laboratories, South San Francisco, Calif.) overnight in a humidified chamber at room temperature. Slides were incubated with biotinylated anti-mouse IgG (Vector Laboratories) at 1:200 for 30 minutes at RT then washed with dPBS and stained with Vectastain Elite ABC Kit (Vector Laboratories) for 30 minutes at RT. Stained slides were washed with dPBS and developed using a Vector NovaRed Substrate Kit (Vector Laboratories) for approximately 1 min. Slides were rinsed in dPBS, followed by a 5 minute wash with tap water, and counterstained with Harris hematoxylin for 45 seconds. The slides were washed with tap water, rinsed with a series of ethanol-xylene washes and mounted with VectaMount permanent mounting medium (Vector Laboratories). The slides were then viewed with a Nikon Eclipse E400 microscope and images were captured using an RT Color Spot camera. The number of Ki67 positive cells were counted and divided by the total number of cells in the basal layer to give a percentage of Ki67 positive cells. At 3 days or 7 days of A/L interface culture on collagen-GAG membranes passively adsorbed with FN, 3 different sections of 5 grafts were evaluated.

FN Cellular Binding Site Detection

To measure the availability of the central cellular binding domain of FN, a monoclonal antibody directed towards this domain (HFN 7.1, Developmental Studies Hybridoma Bank, Iowa City, Iowa) was measured with fluorescence microscopy and image analysis. After passive adsorption or EDC conjugation of FN to CI membranes, the scaffolds were sterilized for cellular culture, and then blocked using 1% heat denatured BSA (in dPBS) for 1 hour at room temperature. HFN 7.1 was added to each well for 1 h in 10% CO₂ at 37° C. Each surface was rinsed in blocking buffer (0.05% Tween-20 (Sigma) and 0.25% BSA in dPBS) and incubated with 546 Alexa Fluor conjugated goat anti-mouse IgG (1:200 in blocking buffer, Molecular Probes, Eugene, Oreg.) for 1 h in 10% CO₂ at 37° C. Slides were then rinsed with dPBS, and images were captured using an RT Color Spot camera. Image J Analysis software was used to determine the relative amount of cellular binding sites in each well. The relative fluorescence intensity was calculated over a region of interest and normalized against fluorescence intensity of non-FN modified membranes. Eight samples were evaluated for collagen-GAG and self-assembled CI membranes that were treated with 100 μg/ml of FN using EDC conjugation or passive adsorption strategy. For self-assembled CI membranes treated with 30 or 300 μg/ml of FN, 4 samples were evaluated. Results are reported as averages and standard deviations and each experiment was repeated twice.

Statistical Analyses

Sigma Stat Version 3.10 (Systat Software Inc., Richmond, Calif.) was used to determine statistical differences among the means of experimental groups. To determine if the means of two different samples were significantly different, a Student's t-test was performed when the samples were drawn from a normally distributed population with equal variance. Sigma Stat uses the Kolmogorov-Smirnov test to test for a normally distributed population and a P value>0.05 indicates normality. For all parametric tests, Sigma Stat assumes equal variance. When the data was not drawn from a normally distributed population (P value<0.05), a Mann-Whitney Rank Sum Test was used and a Levene Median test was used to determine equal variance with a P value>0.05 indicating equal variance. For both the Student's t-test and the Mann-Whitney Rank Sum Test, a p value<0.05 indicated a significant difference between the means of experimental groups.

To determine statistical differences among the means of three or more experimental groups a One Way Analysis of Variance (ANOVA) was used when the samples were drawn from a normally distributed population with equal variance (Kolmogorov-Smirnov test for normal distribution and equal variance was assumed). When the data was not normally distributed, a Kruskal-Wallis One way ANOVA on ranks was performed (Levene Median test to determine equal variance with a P>0.05 indicating equal variance). When a statistical difference was detected among the group means, a Tukey post-hoc analysis was performed for both the One Way ANOVA and Kruskal-Wallis One Way ANOVA on ranks. A p value<0.05, for both variance tests, indicated a significant difference between the groups.

Production of Dermal Scaffold Containing a Microfabricated Basal Lamina Analog

Photolithography of a Master Pattern and Negative Replicates

To mimic the microtopography found at the DEJ, photolithography was used. Master patterns consisting of parallel, three-dimensional channels with widths of 50-400 μm and depth of 200 μm were designed using Pro/Engineer software (PTC, Needham, Mass.). The two dimensional drawing was then printed onto acetate film (CAD/Art Service Inc, Poway, Calif.) with a high resolution laser photoplotter (7008MF: 20,000 dots/inch, Orbotech, Billerica, Mass.). The transparency masks were then aligned on the surface of silicon wafers coated with 200 μm thickness of SU-8 photoresist (Microchem Co., Newton, Mass.) and exposed to a collimated beam of UV light. The wafer was immersed in propylene glycol methyl ether acetate (PGMEA; SU-8 Developer, Microchem Co.) and the unexposed regions were dissolved, leaving a three-dimensional pattern on the silicon wafer (FIG. 11A). To create negative replicate molds, polydimethylsiloxane silicone elastomer (PDMS, Sylgard 184, Dow Corning Corp., Midland, Mich.) was poured onto the surface of the wafer (10:1 base to curing agent), degassed to remove trapped air bubbles, and allowed to polymerize for 2 hours at 65° C. The PDMS was then carefully separated from the silicon wafer (FIG. 11B).

Purification of CI

Acid-soluble type I collagen (CI) was extracted from rat tail tendons using protocols previously described. Rat tails were received from animals that were euthanized for other protocols, which were approved by Worcester Polytechnic Institute, Worcester, Mass., Institutional Animal Care and Use Committee. Briefly, rat tail tendons were extracted from the tails of 13 Sprague Dawley rats, rinsed in dPBS (Hyclone, Logan, Utah), and dissolved in 1600 ml of 3% acetic acid (EMD Chemicals, Inc., Gibbstown, N.J.) at 4° C. overnight. The resulting solution was centrifuged at 8590 rpm for 2 hours and 320 ml of a 30% NaCl (Sigma, St. Louis, Mich.) solution was dripped into the supernatant at 4° C. The resulting solution was allowed to sit for at least 1 hour at 4° C. without disruption and then centrifuged at 4690 rpm for 30 minutes to separate precipitated and liquid material. The precipitated material was resuspended in 400 ml of 0.6% acetic acid and dialyzed for 4 hours against 1 mM HCl (J T Baker, Phillipsburg, N.J.) and the dialysis solution was changed every 4 hours until a clear collagen solution was obtained. This solution was lyophilized and stored in a sealed container at 4° C., until use. Lyophilized collagen was dissolved in 5 mM HCl to obtain a working solution of 10 mg/ml. To produce self-assembled CI membranes, 800 μl of the soluble CI solution was neutralized using 200 μl of 5× Dulbecco's Modified Eagle's Medium (DMEM, Invitrogen, Carlsbad, Calif.) with 0.22 M NaHCO3 and 40 μl of 0.1 M NaOH (Sigma) at 37° C. for 18 hours on circular PDMS molds (FIG. 11C).

Dermal Scaffold Production

To create dermal scaffolds, a collagen-GAG coprecipitate containing collagen (5 mg/ml) and GAG (0.18 mg/ml) was prepared by placing lyophilized bovine hide derived collagen (Semed-S, Kensey Nash Corp., Exton, Pa.) in acetic acid and homogenizing (20,000 rpm) at 4° C. for 90 minutes resulting in a bovine derived collagen suspension. Shark cartilage chondroitin 6-sulfate (Sigma) was dripped into the blending collagen dispersion and blended for an additional 90 minutes. Once fully blended, the collagen-GAG coprecipitate was degassed by centrifugation. Dermal scaffolds were created by placing 20 ml of the collagen-GAG suspension in 70 mm diameter aluminum weigh boats (VWR, West Chester, Pa.) and freezing at −80° C. for 1 hour. Following the initial freezing, the tins were placed in a freeze dryer (Virtis Advantage, Virtis, Inc., Gardner, N.Y.) pre-frozen to −45° C. then lyophilized overnight at a vacuum of 100 mtorr. Following lyophilization, the scaffolds were covalently crosslinked by thermal dehydration (DHT) at 105° C. in a vacuum of less than 200 mtorr for 48 hours. Scaffolds were cut into rectangles approximately 7 cm² (2.5 cm-width×3 cm height) in area and placed in desiccator until use.

Production of Dermal Scaffolds with Microfabricated Basal Lamina Analogs

The production of dermal scaffolds with microfabricated basal lamina analogs began with the fabrication of a self-assembled CI membrane. Initially, a microfabricated self-assembled CI membrane was made by neutralizing 800 μl of 10 mg/ml CI using 200 μl of 5× DMEM containing 0.22 M NaHCO3 and 40 μl of 0.1 M NaOH (Sigma) at 37° C. for 18 hours on PDMS molds containing the negative replicate of the desired channel topography (molds 9.85 cm²) (FIG. 11C). After incubation, 400 μl of 10 mg/ml of CI was neutralized using 100 μl of 5× DMEM containing 0.22 M NaHCO3 and placed directly on the self-assembled CI membrane, and gently spread to cover the entire surface area. Immediately following this step, a precut lyophilized dermal scaffold was placed on top of the neutralizing CI and then incubated at 37° C. for 2 hours to facilitate complete self-assembly of the CI and lamination of the dermal scaffold to the basal lamina analog (FIG. 11D).

FN Conjugation to Microfabricated Basal Lamina Analogs Laminated to Dermal Scaffolds

Carbodiimide 1-ethyl-3-(3-dimethylaminopropyl)carbodiimide hydrochloride (EDC, Sigma) was used to covalently conjugate FN to the surface of the microfabricated basal lamina analog as well as chemically crosslink the basal lamina analog and dermal scaffold. Previously we have reported that this method increases cellular binding site availability of FN. Using protocols described previously, the mole ratio of 5:1 of EDC to carboxylic acid groups in CI was used. Theoretical calculations which estimated the amount of COOH in 1000 amino acids of collagen were used to make the assumption that 1 g of CI contains 1.2 mmol COOH based on amino acid composition of CI. Each dermal scaffold containing a microfabricated basal lamina contains 30 mg of CI for a total of 0.036 mmol COOH, thus requiring 0.18 mmol of EDC. This amount of EDC was dissolved in 50 mM MES (Sigma), prepared in 40% ethanol (Pharmco Products, Inc., Brookfield, Conn.) at a pH of 5.5, and 3 mls of the solution was placed on the dermal scaffold containing a microfabricated basal lamina analog for 4 hours at room temperature (FIG. 11E). Dermal scaffolds containing microfabricated basal lamina analogs were then removed from the EDC solution and immediately placed into air/liquid (A/L) interface culture devices and FN (BD Biosciences, Bedford, Mass.) at 100 μg/ml was placed in the well created on the surface of the collagen membrane over night at room temperature (FIG. 11F). Control dermal scaffolds containing microfabricated basal lamina analogs without FN conjugation were also prepared. These controls received EDC and dPBS instead of FN.

Preparation of De-Epithelialized Acellular Dermis

Following methods previously described by Hamoen et al., De-epithelialized acellular dermis (DED) was prepared to use as a control tissue scaffold. Cadaver skin was obtained from New England Eye and Tissue Transplant Bank and washed 3 times in sterile dPBS. From this point on, sterile conditions were maintained. The cadaver skin was placed in an antibiotic cocktail containing 1× DMEM with 10 μg/ml Ciprofloxacin (Sigma), 2.5 μg/ml Amphoteracin B, 100 U/ml Penicillin, 100 μg/ml Streptomycin, and 100 μg/ml Gentamycin (Invitrogen) and kept at 4° C. for 1 day. The following day, the skin was transferred to a cryopreservation solution composed of 1× DMEM with 15% glycerol (J. T. Baker) and placed at 4° C. for 2 hours. Following this step, skin was placed in sterile mesh gauze soaked in cryopreservation solution and wrapped in sterile aluminum pouches and plastic. Wrapped packages of skin were transferred to −20° C. for 24 hours, and then moved to −80° C. for long term storage.

To prepare the skin for tissue culture, pouches containing cryopreserved tissue were immersed in a tub of water at 15-20° C. until skin was pliable, then refrozen rapidly in liquid nitrogen. This freeze-thaw cycle was repeated 3 times to devitalize the cells. Skin was removed from pouches and washed 3 times in DMEM then placed in antibiotic cocktail for 1 week at 4° C. After 1 week, the skin was transferred into new antibiotic cocktail and incubated for 1 week at 37° C. At the end of the incubation, the epidermis was separated from the dermis, and the dermis was placed into fresh antibiotic cocktail for 4 weeks at 4° C. After 4 weeks, the DED was ready for tissue culture.

In vitro Culture of Dermal Scaffolds Containing Microfabricated Basal Lamina Analogs

Sterilization of Dermal Scaffolds Containing Microfabricated Basal Lamina Analogs

Air/liquid culture devices containing dermal scaffolds with microfabricated basal lamina analogs were placed in sterile containers in 60 ml of 70% ethanol for 1 hour in a laminar flow hood. After 1 hour, devices were transferred to new sterile containers and were rinsed 3 times for 10 minutes each in sterile dPBS and then left overnight in dPBS under sterile conditions.

Culture of Neonatal Human Keratinocytes

Neonatal keratinocytes were cultured as previously described. Neonatal foreskins were obtained from non-identifiable discarded tissues from UMass Memorial Medical Center, Worcester, Mass. and were approved with exempt status from the New England Institutional Review Board. Keratinocyte isolations were performed using an enzymatic treatment with a dispase (Gibco, Gaithersburg, Md.) solution. The cells were propagated on a feeder layer of 3T3-J2 mouse fibroblasts (generously donated by Dr. Stelios Andreadis, State University of New York at Buffalo, Buffalo, N.Y.) and cultured according to methods previously described using keratinocyte media consisting of a 3:1 mixture of DMEM (high glucose) and Ham's F-12 medium (Invitrogen) supplemented with 10% fetal bovine serum (FBS, Hyclone), 10⁻¹⁰ M cholera toxin (Vibrio Cholerae, Type Inaba 569 B), 5 μg/ml transferrin, 0.4 μg/ml hydrocortisone (Calbiochem, La Jolla, Calif.), 0.13 U/ml insulin, 1.4*10⁻⁴ M adenine, 2*10⁻⁹ M triiodo-L-thyronine (Sigma), 1% Penicillin/Streptomycin (Invitrogen), and 0.01 μg/ml epidermal growth factor (EGF, BD Biosciences). After 5 days of culture, keratinocytes were detached using 0.05% Trypsin-EDTA (Invitrogen) and passage 2-3 keratinocytes, from multiple donors were used in all experiments.

Culture of Dermal Scaffolds Containing Microfabricated Basal Lamina Analogs

After sterilization of dermal scaffolds with microfabricated basal lamina analogs, A/L interface culture devices were transferred to sterile 6 well plates for immediate cell culture. Dermal scaffolds with microfabricated basal lamina analogs were preconditioned with seeding media consisting of 3:1 mixture of 1× DMEM (high glucose) and Ham's F-12 medium supplemented with 10⁻¹⁰ M cholera toxin, 0.2 μg/mL hydrocortisone (Calbiochem), 5 μg/mL insulin, 50 μg/mL ascorbic acid (Sigma), and 100 IU/mL and 100 μg/mL penicillin-streptomycin. Keratinocytes were seeded using this media at 500,000 cells/cm² and allowed to adhere for 2 hours in 10% CO₂ at 37° C. After 2 hours, seeding media containing 1% FBS was placed in each well, completely submerging the bioengineered skin substitutes. After 24 h, the keratinocyte seeding medium was removed, and the bioengineered skin substitutes were submerged for an additional 48 h in a keratinocyte priming medium composed of keratinocyte seeding medium (with FBS) supplemented with 24 μM bovine serum albumin (BSA), 1.0 mM L-serine, 10 μM L-carnitine, and a mixture of fatty acids including 25 μM oleic acid, 15 μM linoleic acid, 7 μM arachidonic acid, and 25 μM palmitic acid (Sigma). After 48 h in priming medium, the bioengineered skin substitutes were cultured for 3 or 7 days with an air liquid interface medium composed of serum-free keratinocyte priming medium supplemented with 1.0 ng/mL EGF (FIG. 11G). As controls, composites without FN treatment and DED were cultured in parallel using the same process; however, DED was not sterilized, but placed directly into sterile A/L interface culture devices and keratinocytes were seeded on the papillary surface.

Quantitative Morphometric Analysis of Microfabricated Features of Basal Lamina Analogs

The morphology of the microtopographical features of the surfaces of the basal lamina analogs were evaluated using histology coupled with quantitative image analysis. The specified values for the channels were 200 μm depth and 50 μm, 100 μm, 200 μm, and 400 μm widths. To measure the surface features of the basal lamina analogs, a series of unseeded dermal scaffolds containing microfabricated basal lamina analogs were fixed with 10% buffered formalin solution (EMD Chemicals), dehydrated with increasing concentrations of ethanol, cleared with sec-butyl alcohol (EMD Chemicals), and embedded in Paraplast tissue embedding medium (McCormick Scientific, St. Louis, Mo.). Six micron sections were cut using a Leica RM 2235 (Leica Microsystems, Inc., Bannockburn, Ill.) in a plane perpendicular to the surface of the basal lamina. Sections were mounted on poly-l-lysine coated slides (Erie Scientific Company, Portsmouth, N.H.). Tissue sections were deparaffinized in reverse ethanol-xylene (Pharmco Products, Inc. and EMD Chemicals) washes and stained with Harris Hematoxylin (Richard Allen Scientific, Kalamazoo, Mich.) for 4 minutes. Slides were rinsed with dH2O and 1% acid alcohol and stained with Eosin (Richard Allen Scientific) for 30 seconds. The slides were then cleared in a series of ethanol and xylene and cover slipped using Permount (Fisher Scientific, Hampton, N.H.) mounting medium. Brightfield images were captured of each section using a Nikon Eclipse E400 microscope (Nikon, Inc., Melville, N.Y.) coupled to an RT Color Spot camera (Spot Diagnostics, Sterline Heights, Mich.). For each sample the depths of the channels and the widths of the channels were measured using Image J software (downloaded from http://rsb.info.nih gov.ezproxy.umassmed.edu/ij/). Values are reported as mean +/−SEM.

Analyses of Epithelialization and Regenerative Capacity of Bioengineered Skin Substitutes Containing Microfabricated Basal Lamina Analogs

Epidermal Thickness and Graft Morphology

Epidermal thickness and graft morphology on the surfaces of the basal lamina analogs laminated to dermal scaffolds were evaluated after 3 or 7 days of A/L interface culture. Samples were embedded in paraffin wax, sectioned, and mounted as described previously in the section entitled Quantitative Morphometric Analyses of Microfabricated Features of Basal Lamina Analogs Laminated to Dermal Scaffolds. Paraffin sections were deparaffinized in reverse ethanol-xylene washes and stained with Hematoxylin and Eosin. Brightfield images were captured and using Image J measurements of channel depth, channel widths, and epithelial thickness in each channel. Additionally the epidermal thickness of the flat region adjacent to the channels (papillary plateau) was measured (FIG. 12A insert). Multiple measurements were made for each sample since each sample contained multiple channels. For epithelialized DED and native tissues, the thickness of the epidermal layer was measured in the rete ridges and on the papillary plateaus. The dimensions of the rete ridges were also measured.

To characterize the effect of channel dimensions on epidermal thickness, the epidermal thicknesses were measured in channels with widths that were within +/−2 SEM of the topography validation width measurements, for each specified channel width. Data points were excluded from all other channels from this analysis. These data points were then individually normalized to the depth of their channel. The normalized data from each specified channel width was then averaged and reported as a mean value +/−SEM. Sample values for the 50, 100, 200, and 400 μm width channels were n=5, 5, 6, 11 at 3 days, respectively and n=5, 6, 15, and 13 at 7 days, respectively. At both 3 and 7 days n=4 for DED and n=4 for foreskin tissue.

Keratinocyte Proliferation

Keratinocyte proliferation was evaluated after 3 or 7 days of A/L interface culture by measuring the presence of Ki67, a cell cycle associated antigen. Samples were embedded in paraffin, sectioned, and mounted on Superfrost Plus slides (VWR, West Chester, Pa.) coated with poly-L-lysine (Sigma). The paraffin sections were deparaffinized in reverse ethanol-xylene washes, and the antigens were unmasked by placing the slides in boiling Vector UnMasking solution (Vector Laboratories, Inc, Burlingame, Calif.) in a Manttra pressure cooker (Manttra, Inc., Virginia Beach, Va.) for 1 minute after maximum pressure was achieved. Slides were then incubated with blocking solution (10% normal horse serum (Vector Laboratories) in dPBS) for 10 min at room temperature and then treated with predilute mouse-antihuman Ki67 antibody (Zymed Laboratories, South San Francisco, Calif.) overnight in a humidified chamber (Sigma) at room temperature. Slides were incubated with biotinylated anti-mouse IgG (Vector Laboratories) at 1:200 for 30 minutes at RT. The slides were washed with dPBS and stained with Vectastain Elite ABC Kit (Vector Laboratories) for 30 minutes at room temperature. Slides were washed with dPBS and developed using a Vector NovaRed Substrate Kit (Vector Laboratories) for approximately 1 min for bioengineered skin substitutes and epithelialized DED, and 5 min for native tissues. Slides were rinsed in dPBS, followed by a 5 minute wash with tap water, and counterstained with Harris hematoxylin for 45 s. The slides were washed with tap water and then went through ethanol-xylene washes and mounted with VectaMount permanent mounting medium (Vector Laboratories). The slides were viewed with a Nikon Eclipse E400 microscope and images were captured using an RT Color Spot camera. The number of Ki67 positive basal cells and total basal cell number were counted over the length of the basal lamina in each channel and for control tissues, over the entire image. The data from each specified channel width was averaged and reported as the mean value +/−SEM. Samples for 50, 100, 200, and 400 μm width channels were n=5, 6, 7, and 10 at 3 days, respectively and n=5, 6, 10, and 11 at 7 days, respectively. At both 3 and 7 days of A/L interface culture n=4 for epithelialized DED. Samples for foreskin tissue were n=5. Only one sample of breast control tissue was obtained and 3 images of the sample were evaluated and averaged reported as the mean +/− standard deviations. Breast tissue was not included in statistical analyses.

Beta-1 Analysis of Keratinocyte Colonies

To evaluate keratinocyte expression of β₁ integrins in routine keratinocyte co-culture, we utilized quantitative immunofluorescence staining on tissue culture substrates. For the tissue culture substrates, keratinocytes were cultured in 6 well culture plates, using methods previously described. After 5 days of culture, each well was rinsed with dPBS and treated for 10 minutes with a fixing and permeabilizing solution containing dPBS, 4% formaldehyde (Ted Pella, Redding, Calif.), and 0.2% of Triton X-100 (Sigma). Wells were then rinsed to remove fixative and permeabilizing solution and blocked with a 1% BSA solution in dPBS for 10 minutes. Silicone gaskets made from PDMS with inner diameter of ˜2 cm² were secured in the center of each well using silicone vacuum grease (Dow corning, Midland, Mich.). A primary antibody directed against β₁ (Anti-CD29, BioGenex, San Ramon, Calif.) at a concentration of 1:100 in blocking solution was applied for 2 hours at 37° C. Following incubation, each sample was washed with dPBS twice, 5 minutes each time. Goat anti-mouse (Alexa Fluor 546, Invitrogen) secondary antibody at a dilution of 1:100 in blocking solution was placed in each well and incubated for 1 hour at 37° C. After incubation, the wells were rinsed and Hoeschst nuclear reagent (Invitrogen) was added at 0.06 mM (in dH₂O) for 5 minutes at 37° C. The wells were rinsed with dPBS, the gaskets removed, and the wells were cover slipped using an aqueous mounting medium containing anti-fading agents (Biomeda Corp, Foster City, Calif.). Each image was captured using the same exposure time. Using Image J software, the histogram function was used to determine the greatest fluorescence intensity. Following previously published methods, the greatest fluorescence intensity recorded was subdivided into three regions, the dullest (bottom ⅓), the brightest (top ⅓) and the remaining (middle ⅓). Cells that had intensity values in the top ⅓ around their perimeter were considered integrin-bright. The number of cells that were integrin bright were counted as well as the total number of cells in the colony. The average percent of integrin-bright keratinocytes for 4 separate wells was reported as a mean value +/−SEM since multiple images were captured and analyzed for each well.

Beta-1 Expression in Bioengineered Skin Substitutes, Epithelialized DEDs, and Human Tissue

The expression of β₁ for basal keratinocytes in bioengineered skin substitutes, epithelialized DED, and human tissues, was analyzed using immunohistochemistry and quantitative analyses of fluorescent microscope images. Tissue samples, 6 μm thick, were mounted on Superfrost Plus slides coated with poly-l-lysine. Following the same procedure as for Ki67 detection, all samples were deparaffinized and the antigens were unmasked. The same procedure was then followed as for the analysis of β₁ of keratinocyte colonies on tissue culture plastic, except samples were cover slipped with Vectashield Mounting Medium with DAPI (Vector Laboratories) to visualize nuclei. Human foreskins and breast tissue were obtained from non-identifiable discarded tissues from UMass Memorial Medical Center, Worcester, Mass. and were exempt from New England Institutional Review Board review. The human tissues were processed the same way as the bioengineered skin substitutes and epithelialized DED. Using Image J software, the average relative fluorescence intensity (RFI) value of cell borders was mapped for basal keratinocytes for all tissues evaluated. Previously, it has been determined that β₁ intensities correspond with ESC populations and integrin-bright patches have been used as an indicator of ESC localization areas. Once measured, the average RFI was plotted to evaluate integrin-bright and integrin-dull regions of the basal lamina. Similar to β₁ expression in the colonies, cells that had intensity values in the top ⅓ were considered integrin-bright.

Statistical Analyses

Sigma Stat Version 3.10 (Systat Software Inc., Richmond, Calif.) was used to determine statistical differences among the means of experimental groups. To determine if the means of two different samples were significantly different, a Student's t-test was performed when the samples were drawn from a normally distributed population with equal variance. Sigma Stat uses the Kolmogorov-Smirnov test to test for a normally distributed population and a P value>0.05 indicates normality. For all parametric tests, Sigma Stat assumes equal variance. When the data was not drawn from a normally distributed population (P value<0.05), a Mann-Whitney Rank Sum Test was used and a Levene Median test was used to determine equal variance with a P value>0.05 indicating equal variance. For both the Student's t-test and the Mann-Whitney Rank Sum Test, a p value<0.05 indicated a significant difference between the means of experimental groups.

To determine statistical differences among the means of three or more experimental groups a One Way Analysis of Variance (ANOVA) was used when the samples were drawn from a normally distributed population with equal variance (Kolmogorov-Smirnov test for normal distribution and equal variance was assumed). When the data was not normally distributed, a Kruskal-Wallis One way ANOVA on ranks was performed (Levene Median test to determine equal variance with a P>0.05 indicating equal variance). When a statistical difference was detected among the group means, a Tukey post-hoc analysis was performed for both the One Way ANOVA and Kruskal-Wallis One Way ANOVA on ranks. A p value<0.05, for both variance tests, indicated a significant difference between the groups.

ABBREVIATIONS: ANOVA: Analysis of variance; A/L: Air liquid interface; CI: Type I collagen; CIV: Type W collagen; CEA: Cultured epithelial autografts; CFE: Colony forming efficiency; DED: De-epithelialized acellular dermis; DEJ: Dermal-epidermal junction; DHT: Dehydrothermal; DMEM: Dulbecco's Modified Eagle's Medium; DPBS: Dulbecco's phosphate buffered saline; ECM: Extracellular matrix; EDC: Carbodiimide 1-ethyl-3-(3-dimethylaminopropyl)carbodiimide hydrochloride; ESC: Epidermal stem cell; FA: Focal adhesions; FN: Fibronectin; FACs: Fluorescence activated cell sorting; GAG: Glycosaminoglycan; HFN 7.1: Antibody against central cellular binding domain of fibronectin; HTS: High throughput screening; KCM: Keratinocyte medium; KCM (-S-GF): Serum free growth factor free keratinocyte media; Ki67: Cell cycle associated antigen; LN: Laminin; LRCs: Label retaining cells; MMPs: Matrix metalloproteinases; MTT: Thiazoyl blue tetrazolium bromide; NHK: Neonatal human keratinocytes; SAM: Self-assembled monolayer; PBSABC: Phosphate buffered saline with calcium and magnesium salts; PDMS: Polydimethylsiloxane; PEG: Polyethylene glycol; PHSRN: Proline, histidine, serine, arginine, asparagine; PLGA: Poly(lactic-co-glycolic acid); RGD: Arginine-glycine-aspartic acid; ROI: Region of interest; RTT: Rat tail tendon; TA: Transit amplifying cells

We claim:

1. A skin substitute comprising a basal lamina analog comprising extracellular matrix protein, a dermal sponge and keratinocytes.

2. The skin substitute of claim 1, wherein said extracellular matrix protein is selected from the group consisting of collagen I, collagen IV, fibronectin, laminin, glycosaminoglycan and combinations thereof.

3. The skin substitute of claim 2, wherein fibronectin is covalently bound to collagen I, collagen IV or collagen-glycosaminoglycan.

4. The skin substitute of claim 3, wherein said fibronectin is covalently bound using a chemical crosslinking agent.

5. The skin substitute of claim 4, wherein said chemical crosslinking agent is 1-ethyl-3-(3-dimethylaminopropyl)carbodiimide hydrochloride.

6. A method of making a skin substitute comprising:
a) creating a master pattern containing channels on a silicon wafer;
b) casting polydimethylsiloxane onto the silicon wafer of step (a);
c) allowing the polydimethylsiloxane to polymerize;
d) casting a first extracellular matrix protein onto the polymerized polydimethylsiloxane;
e) allowing the first extracellular matrix protein to polymerize;
f) casting a second extracellular matrix protein onto the back surface of the first extracellular matrix protein;
g) attaching an extracellular matrix protein sponge to the back surface of the first extracellular matrix protein;
h) chemically crosslinking the first extracellular matrix protein, second extracellular matrix protein and extracellular matrix protein sponge to form a composite;
i) removing the composite from the polymerized polydimethylsiloxane;
j) conjugating fibronectin to the front surface of the composite;
j) sterilizing the composite; and
k) adding keratinocytes.

7. The method of claim 6, wherein said first and second extracellular matrix protein are collagen.

8. The method of claim 6, wherein said extracellular matrix protein sponge is collagen-glycosaminoglycan.

9. A method of treating wounds or burns comprising administering a skin substitute comprising a basal lamina analog comprising extracellular matrix protein, a dermal sponge and keratinocytes.
